(19) United States
(12) Patent Application Publication
(10) Pub. No.: US 2024/0255849 A1
(43) Pub. Date: Aug. 1, 2024

(54) RESIST COMPOUND FOR PHOTOLITHOGRAPHY, METHOD FOR FORMING THE SAME, AND METHOD FOR MANUFACTURING SEMICONDUCTOR DEVICES USING THE SAME

(71) Applicant: Inha University Research and Business Foundation, Incheon (KR)

(72) Inventors: Jinkyun LEE, Incheon (KR); Gayoung KIM, Hwaseong-si (KR); Yejin KU, Incheon (KR)

(73) Assignee: Inha University Research and Business Foundation, Incheon (KR)

(21) Appl. No.: 18/463,646

(22) Filed: Sep. 8, 2023

(30) Foreign Application Priority Data
Dec. 27, 2022 (KR) 10-2022-0186255

(51) Int. Cl. G03F 7/004; C07C 41/09; C07F 7/18; H01L 21/027

(52) U.S. Cl. CPC G03F 7/0045; C07C 41/09; C07F 7/1804; C07F 7/1888; H01L 21/0274

(57) ABSTRACT

Provided are a resist compound for photolithography, a method for forming the same, and a method for manufacturing a semiconductor device using the same. The resist compound is represented by Formula 1.

### CROSS-REFERENCE TO RELATED APPLICATIONS

This U.S. non-provisional patent application claims priority under 35 U.S.C. § 119 of Korean Patent Application No. 10-2022-0186255, filed on Dec. 27, 2022, the entire contents of which are hereby incorporated by reference.

### BACKGROUND

The present disclosure herein relates to a resist compound for photolithography used for the manufacture of semiconductor devices, a method for forming the same, and a method for manufacturing semiconductor devices using the same.

Photolithography may include an exposing process and a developing process. The exposing process may include exposing a resist layer to a specific wavelength of light to induce the change of the chemical structure of the resist layer. The developing process may include selectively removing of the exposed part or the unexposed part of the resist layer by using a solubility difference between the exposed part and the unexposed part.

Recently, as semiconductor devices are highly integrated and downsized, line widths of patterns in semiconductor devices are miniaturized. In order to form minute patterns, various studies are conducted to improve resolution and sensitivity of resist patterns formed by photolithography and to restrain the collapse of resist patterns. Further, a desire for a resist pattern having excellent etching resistance to an etching process is being increased.

### SUMMARY

The technical task for accomplishing of the present disclosure is to provide a resist compound which may improve the resolution and sensitivity of a photoresist pattern and increasing the etching resistance of the photoresist pattern, a method for forming the same, and a method for manufacturing a semiconductor device using the same.

Another technical task for accomplishing of the present disclosure is to provide a resist compound which may restrain the collapse of a photoresist pattern, a method for forming the same, and a method for manufacturing a semiconductor device using the same.

The task for solving of the present disclosure is not limited to the aforementioned tasks, and unreferred other tasks may be clearly understood by a person skilled in the art from the description below.

According to an embodiment of the inventive concept, there is provided a resist compound for photolithography, represented by Formula 1.

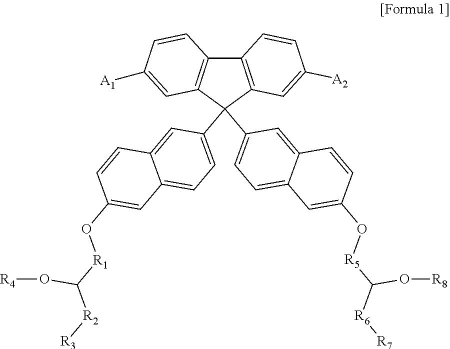

In Formula 1, R₁ and R₅ are each independently an alkylene group of 1 to 4 carbon atoms, R₂ and R₆ are each independently a single bond or an alkylene group of 1 to 4 carbon atoms, R₃ and R₇ are each independently a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms or a fluoroaryl group of 6 to 20 carbon atoms, R₄ and R₈ are each independently hydrogen, deuterium, or a vinyl silyl group, and A₁ and A₂ are hydrogen or an aryl group of 6 to 20 carbon atoms.

According to another embodiment of the inventive concept, there is provided a method for forming a resist compound for photolithography, including: providing a single molecule having a core structure including at least six benzene rings; and combining a functional group with the core structure. The combining of the functional group with the core structure includes combining a preliminary functional group including a fluorine-containing group and a hydroxyl group (—OH) with the core structure. The combining of the preliminary functional group with the core structure includes performing S_{N}2 type ring-opening reaction using the single molecule having the core structure, and an oxygen-containing heterocyclic compound of 2 to 5 carbon atoms, having the fluorine-containing functional group. The preliminary functional group is combined with any one among the benzene rings of the core structure.

According to another embodiment of the inventive concept, there is provided a method for manufacturing a semiconductor device, including: forming a lower layer on a substrate; and forming a photoresist layer on the lower layer. The photoresist layer includes a resist compound having an organic single molecular structure. The resist compound includes a core structure including at least six benzene rings, and a functional group combined with at least one of the benzene rings of the core structure. The functional group includes at least one among a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, a fluoroaryl group of 6 to 20 carbon atoms, and a vinyl silyl group.

### BRIEF DESCRIPTION OF THE FIGURES

The accompanying drawings are included to provide a further understanding of the inventive concept, and are incorporated in and constitute a part of this specification. The drawings illustrate example embodiments of the inventive concept and, together with the description, serve to explain principles of the inventive concept. In the drawings:

FIG. 1A is a graph showing the nuclear magnetic resonance spectrum results of a resist compound (HNF-OH) synthesized according to Experimental Example 1-1;

FIG. 1B is a graph showing the nuclear magnetic resonance spectrum results of a resist compound (HNF-OH(s)) synthesized according to Experimental Example 1-2;

FIG. 1C and FIG. 1D are graphs showing the nuclear magnetic resonance spectrum results of DPF and DPNF, synthesized according to Experimental Example 1-3;

FIG. 1E is a graph showing the nuclear magnetic resonance spectrum results of a resist compound (DPNF-OH) synthesized according to Experimental Example 1-4;

FIG. 2A is a graph showing the nuclear magnetic resonance spectrum results of a resist compound (HNF-DVS) synthesized according to Experimental Example 2-1;

FIG. 2B is a graph showing the nuclear magnetic resonance spectrum results of a resist compound (HNF-DVS(s)) synthesized according to Experimental Example 2-2;

FIG. 2C is a graph showing the nuclear magnetic resonance spectrum results of a resist compound (DPNF-DVS) synthesized according to Experimental Example 2-3;

FIG. 3 to FIG. 6 are cross-sectional views showing a method for manufacturing a semiconductor device using a resist compound according to embodiments of the inventive concept;

FIG. 7 shows scanning electron microscope images of photoresist patterns formed according to Experimental Example 3;

FIG. 8 is a graph showing the solubility evaluation results of a photoresist thin layer formed according to Experimental Example 3;

FIG. 9 is a graph showing the solubility evaluation results of a photoresist thin layer according to Experimental Example 4;

FIG. 10 shows a scanning electron microscope image of a photoresist pattern formed according to Experimental Example 5; and

FIG. 11 is a graph comparing the etching rates of photoresist thin layers formed according to Experimental Example 6.

### DETAILED DESCRIPTION

Preferred embodiments of the inventive concept will be explained with reference to the accompany drawings for sufficient understanding of the configurations and effects of the inventive concept. The inventive concept may, however, be embodied in various forms, have various modifications and should not be construed as limited to the embodiments set forth herein. The embodiments are provided to complete the disclosure of the inventive concept through the explanation of the embodiments and to completely inform a person having ordinary knowledge in this technical field to which the inventive concept belongs of the scope of the inventive concept. A person having ordinary skill in this technical field may understand which environment is suitable for performing the inventive concept.

The terminology used herein is for the purpose of describing example embodiments and is not intended to limit the inventive concept. In the description, the singular forms are intended to include the plural forms as well, unless the context clearly indicates otherwise. It will be further understood that the terms “comprises” and/or “comprising,” when used in this specification, specify the presence of stated materials, constituent elements, steps, operations and/or devices, but do not preclude the presence or addition of one or more other stated materials, constituent elements, steps, operations and/or devices.

In the present description, an alkyl group includes a monovalent saturated hydrocarbon group of a linear chain, branched chain or cyclic chain, unless otherwise indicated.

In the present description, an alkylene group includes a divalent saturated hydrocarbon group of a linear chain, branched chain or cyclic chain, unless otherwise indicated.

In the present description, fluoroalkyl is an alkyl group of which at least one hydrogen is substituted with fluorine, fluoroalkylene is an alkylene group of which at least one hydrogen is substituted with fluorine, fluoroaryl is an aryl group of which at least one hydrogen is substituted with fluorine, and alkoxy fluoride is an alkoxy group of which at least one hydrogen is substituted with fluorine.

In the description, a hydrocarbon group “having a substituent” means that at least a portion of the hydrogen atoms of the hydrocarbon group are substituted with functional groups or atoms, other than hydrogen atoms.

In the description, a case of not drawing a chemical bond at a position where a chemical bond is required may mean that a hydrogen atom is bonded at the position, unless otherwise defined.

Hereinafter, embodiments of the inventive concept will be explained in detail with reference to attached drawings. The same reference numerals are used for the same constituent elements on the drawings, and repeated explanation thereon will be omitted.

### A Resist Compound According to Embodiments of the Inventive Concept Will be Explained.

The resist compound according to embodiments of the inventive concept may be used for the manufacture of a semiconductor device and may be used in a photolithography process for the manufacture of a semiconductor device. The resist compound may be used, for example, in an extreme ultraviolet or e-beam lithography process. The extreme ultraviolet may mean ultraviolet having a wavelength of about 10 nm to about 124 nm, in detail, a wavelength of about 13.0 nm to about 13.9 nm, in more detail, a wavelength of about 13.4 nm to about 13.6 nm.

The resist compound may include an organic single molecular structure. The resist compound may include a core structure and a functional group combined with the core structure. The core structure may be a single molecule or a dendrimer type. In an embodiment, the core structure may include a compound of at least six hydrocarbon rings, in detail, at least six benzene rings. The functional group may be combined with any one among the benzene rings. The functional group may include at least one among a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, a fluoroaryl group of 6 to 20 carbon atoms, or a vinyl silyl group.

The resist compound may be represented by Formula 1.

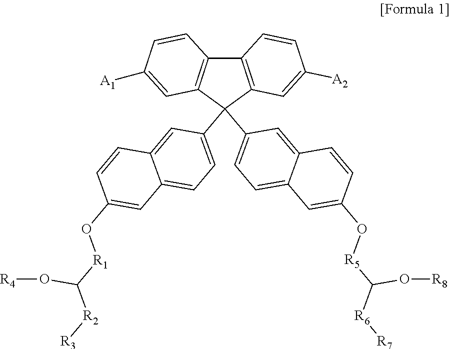

In Formula 1, R₁ and R₅ are each independently an alkylene group of 1 to 4 carbon atoms, and R₂ and R₆ are each independently a single bond or an alkylene group of 1 to 4 carbon atoms. R₃ and R₇ are each independently a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms or a fluoroaryl group of 6 to 20 carbon atoms. R₄ and R₈ are each independently hydrogen, deuterium, or a vinyl silyl group, and A₁ and A₂ are hydrogen or an aryl group of 6 to 20 carbon atoms.

R₁, R₂, R₅ and R₆ may each independently have a structure of —CₙH₂ₙ—, and “n” may be an integer of 1 to 4.

R₃ and R₇ may be each independently a functional group represented by Formula 2-1, Formula 2-2, Formula 2-3, or Formula 2-4.

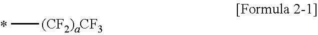

In Formula 2-1, “a” is an integer of 1 to 19.

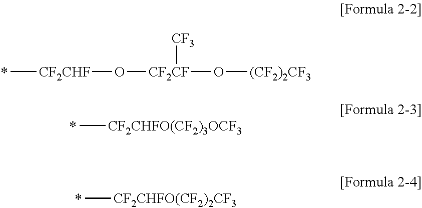

In Formula 2-1 to Formula 2-4, * is a part bonded to the carbon of Formula 1.

R₄ and R₈ may be each independently a functional group represented by Formula 3.

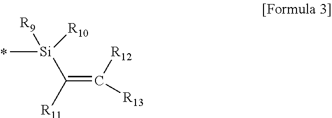

In Formula 3, R₉, R₁₀, R₁₁, R₁₂ and R₁₃ are each independently hydrogen, deuterium, or an alkyl group of 1 to 3 carbon atoms, and * is a part bonded to the oxygen of Formula 1.

The functional group represented by Formula 3 may include a functional group represented by Formula 3-1.

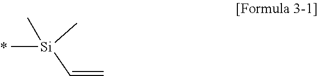

In Formula 3-1, * is a part bonded to the oxygen of Formula 1.

A₁ and A₂ may be each independently a phenyl group or a phenyl group having a substituent. The phenyl group having the substituent means that at least a portion of the hydrogen atoms of the phenyl group are substituted with functional groups or atoms other than the hydrogen atom. The substituent may be an alkyl group (—R) of 1 to 20 carbon atoms, a fluoroalkyl group of 1 to 20 carbon atoms, a hydroxyl group (—OH), an alkoxy group (—OR) of 1 to 20 carbon atoms, a fluoroalkoxy group of 1 to 20 carbon atoms, a thiol group (—SH), an alkoxythiol group (—SOR) of 1 to 20 carbon atoms, a fluoroalkoxy thiol group of 1 to 20 carbon atoms, a carboxyl group (—COOH), an alkylcarbonyl group (—COR) of 1 to 20 carbon atoms, a fluoroalkyl carbonyl group of 1 to 20 carbon atoms, an alkyl ester group (—COOR) of 1 to 20 carbon atoms, a fluoroalkyl ester group of 1 to 20 carbon atoms, an amino group (NH₂), an alkylamino group (—NR₂) of 1 to 20 carbon atoms, a fluoroalkyl amino group of 1 to 20 carbon atoms, a nitro group (—NO₂), or a halogen atom (—F, —Cl, —Br or —I). In an embodiment, the substituent may be *—CF₃, *—OCH₃ or *—O(CF₂)₅CF₃, where * is a part bonded to the carbon of the phenyl group.

According to some embodiments, the resist compound represented by Formula 1 may include a material represented by Formula 4 or Formula 5.

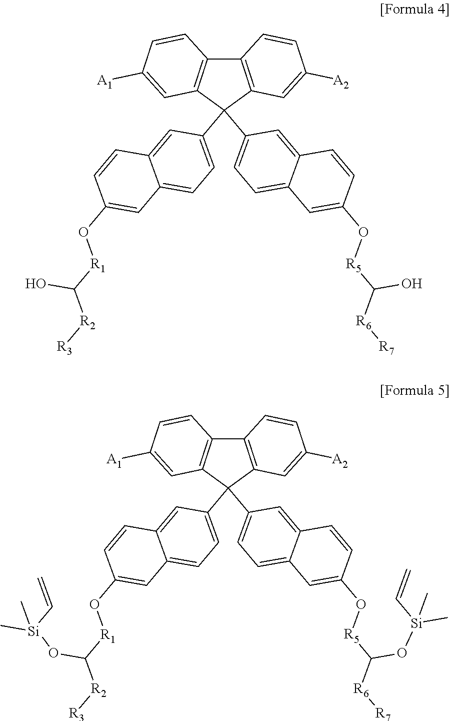

In Formula 4 and Formula 5, R₁, R₂, R₃, R₅, R₆, R₇, A₁ and A₂ are the same as defined in Formula 1.

The resist compound represented by Formula 4 may include a material represented by Formula 4-1, Formula 4-2, Formula 4-3 or Formula 4-4.

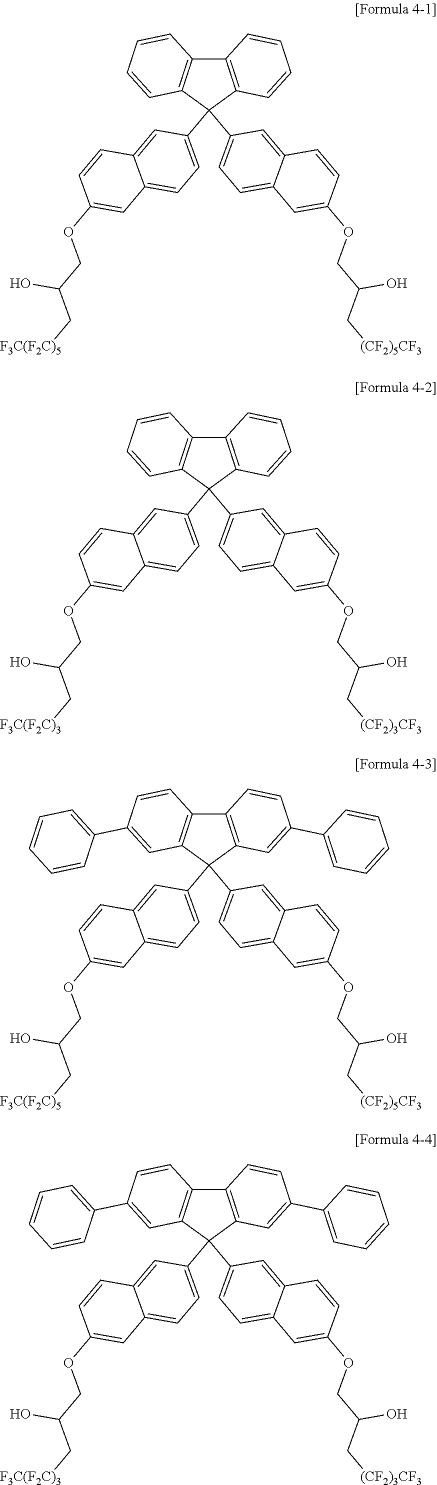

The resist compound represented by Formula 5 may include a material represented by Formula 5-1, Formula 5-2, Formula 5-3 or Formula 5-4.

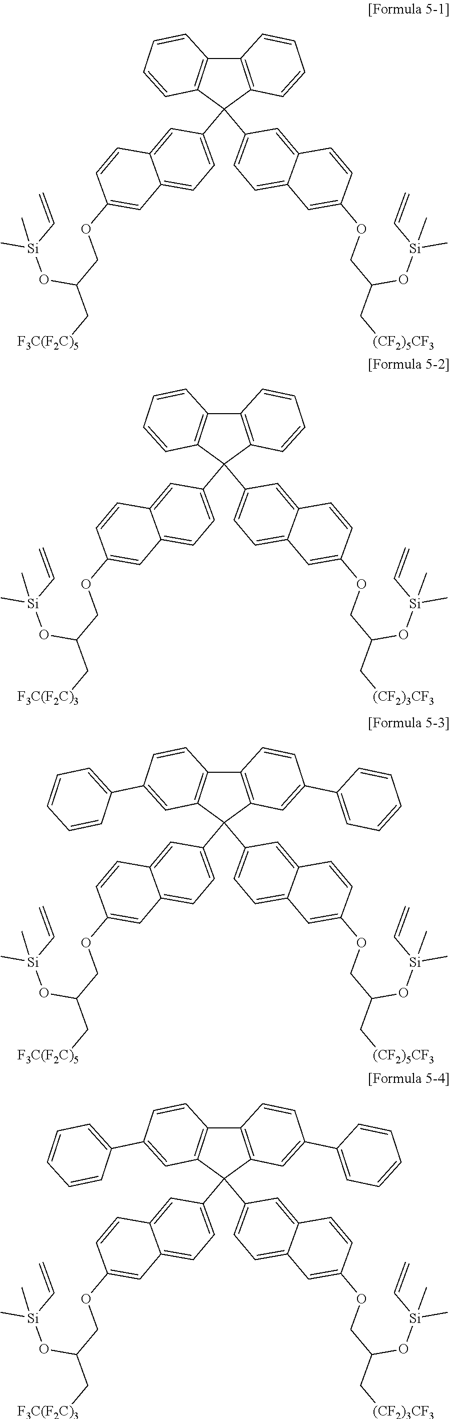

### The Method for Forming a Resist Compound According to Embodiments of the Inventive Concept Will be Explained.

The method for forming the resist compound may include providing a single molecule having a core structure and combining a functional group with the core structure.

The core structure may have a dendrimer type and may include, for example, a compound of at least six hydrocarbon rings, in detail, at least six benzene rings. The functional group may be combined with any one among the benzene rings of the core structure.

The single molecule having the core structure may include a material represented by Formula 6-1 or Formula 6-2.

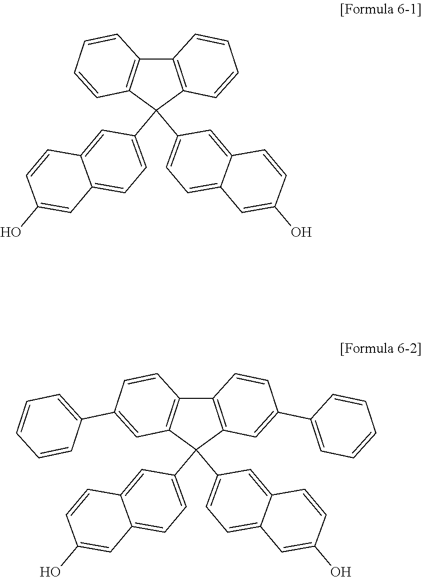 [SMILES: Oc1ccc2cc(C3(c4ccc5cc(O)ccc5c4)c4ccccc4-c4ccccc43)ccc2c1 ; Oc1ccc2cc(C3(c4ccc5cc(O)ccc5c4)c4cc(-c5ccccc5)ccc4-c4ccc(-c5ccccc5)cc43)ccc2c1]

The combination of the functional group with the core structure may include combining a preliminary functional group including a fluorine-containing functional group and a hydroxyl group (—OH) with the core structure. The fluorine-containing functional group may be a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, or a fluoroaryl group of 6 to 20 carbon atoms. The fluorine-containing functional group may be represented by Formula 2-1, Formula 2-2, Formula 2-3 or Formula 2-4.

The combination of the preliminary functional group with the core structure may include performing S_{N}2 (bimolecular nucleophilic substitution) type ring-opening reaction using the single molecule having the core structure and an oxygen-containing heterocyclic compound of 2 to 5 carbon atoms, having the fluorine-containing functional group. For example, the oxygen-containing heterocyclic compound may be an epoxide.

Particularly, the combination of the preliminary functional group with the core structure may include preparing an epoxide having the fluorine-containing functional group according to Reaction 1, and performing S_{N}2 type ring-opening reaction using the single molecule having the core structure and the epoxide having the fluorine-containing functional group according to Reaction 2-1 and Reaction 2-2.

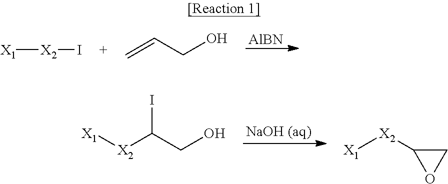

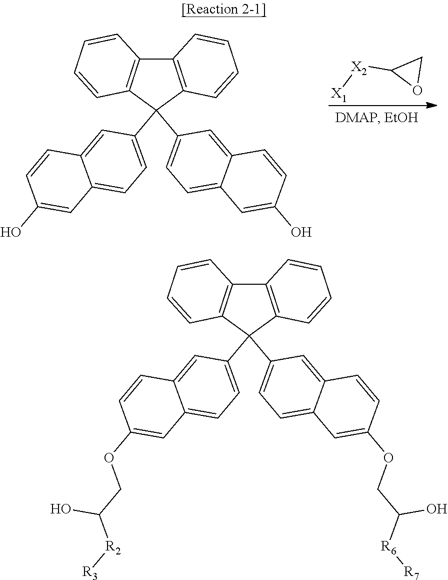

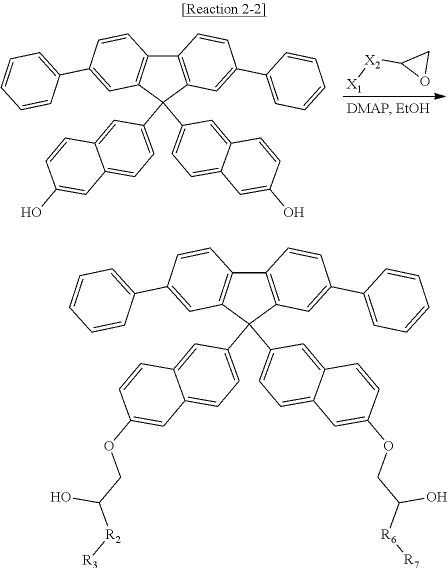

In Reaction 1, Reaction 2-1 and Reaction 2-2, X₁, R₃ and R₇ are the fluorine-containing functional groups. X₁ is a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, or a fluoroaryl group of 6 to 20 carbon atoms. X₁ may be a functional group represented by Formula 2-1, Formula 2-2, Formula 2-3 or Formula 2-4. X₂ may be a single bond or an alkylene group of 1 to 4 carbon atoms. R₂, R₃, R₆ and R₇ are the same as defined in Formula 1. In Reaction 1, AIBN is azobis-isobutyronitrile. In Reaction 2-1 and Reaction 2-2, DMAP is 4-dimethylaminopyridine, and EtOH is ethanol.

Reaction 1 may include Reaction 1-1 or Reaction 1-2.

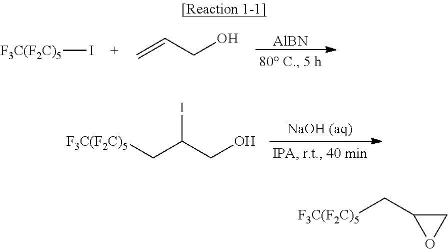

In Reaction 1, IPA is isopropyl alcohol.

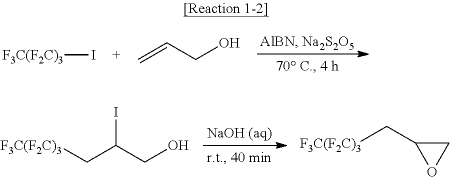

Reaction 2-1 may include Reaction 2-1A or Reaction 2-1B.

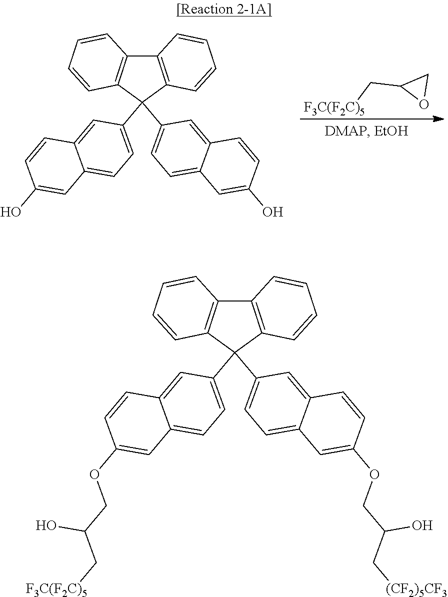

By Reaction 2-1A, the resist compound represented by Formula 4-1 may be produced.

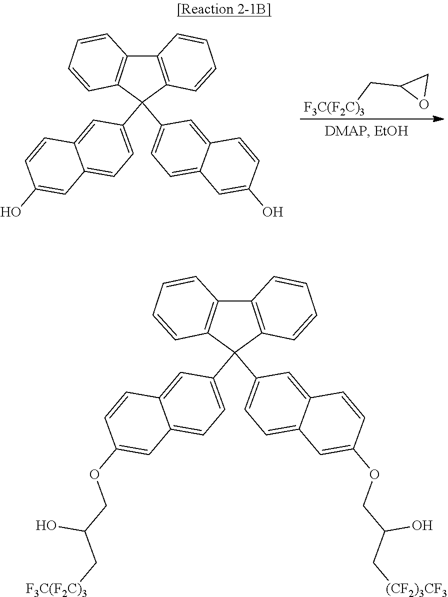

By Reaction 2-1B, the resist compound represented by Formula 4-2 may be produced.

Reaction 2-2 may include Reaction 2-2A or Reaction 2-2B.

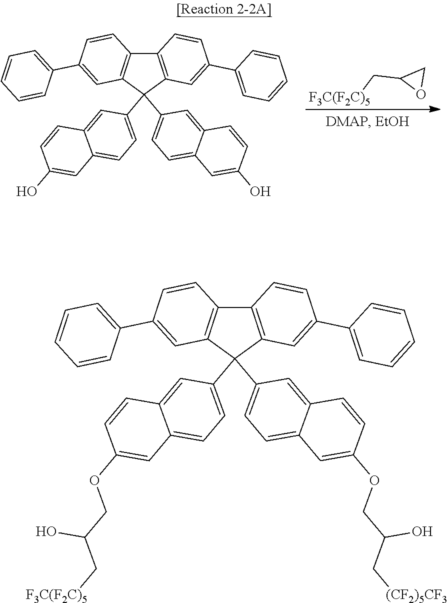

By Reaction 2-2A, the resist compound represented by Formula 4-3 may be produced.

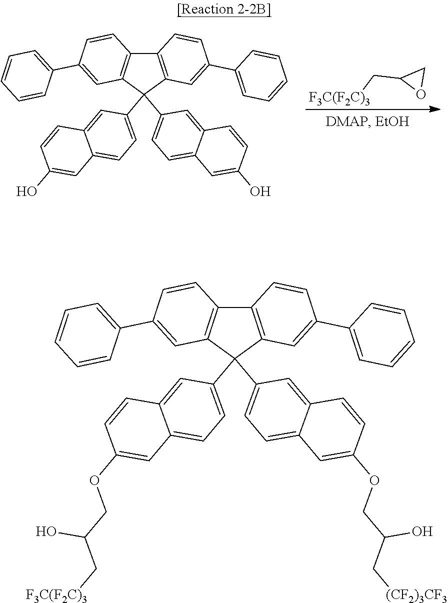

By Reaction 2-2B, the resist compound represented by Formula 4-4 may be produced.

The combination of the functional group with the core structure, may further include substituting the hydroxyl group (—OH) of the preliminary functional group with a vinyl silyl group. The vinyl silyl group may be represented by Formula 3. The substitution of the hydroxyl group (—OH) of the preliminary functional group with the vinyl silyl group may be performed according to Reaction 3-1 or Reaction 3-2.

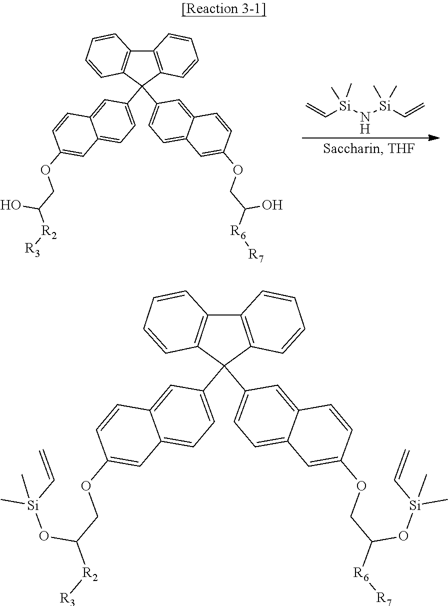

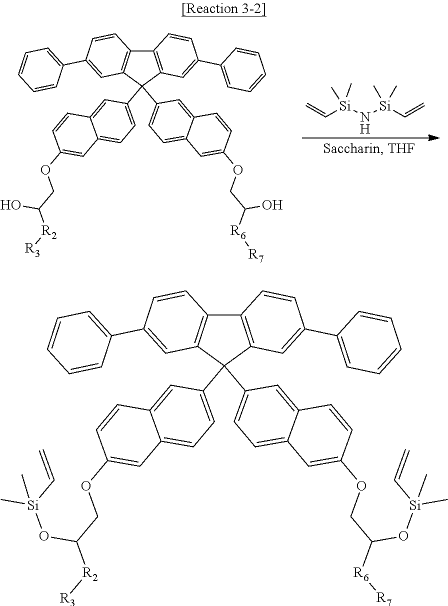

In Reaction 3-1 and Reaction 3-2. R₂, R₃, R₆ and R₇ are the same as defined in Reaction 1. THF is tetrahydrofuran.

Reaction 3-1 may include Reaction 3-1A or Reaction 3-1B.

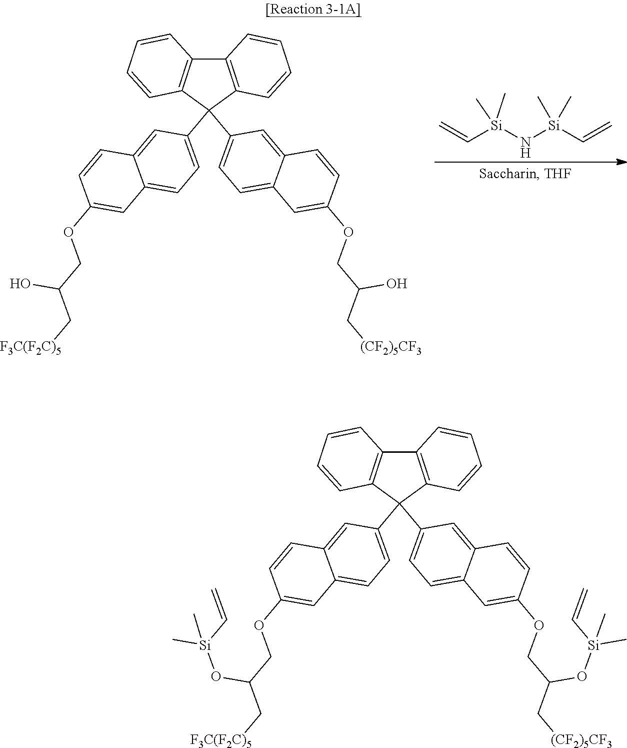

By Reaction 3-1A, the resist compound represented by Formula 5-1 may be produced.

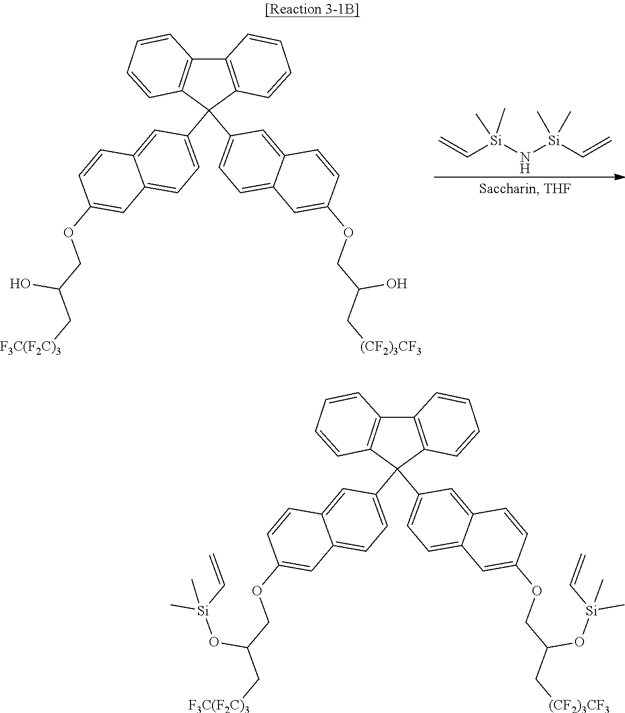

By Reaction 3-1B, the resist compound represented by Formula 5-2 may be produced.

Reaction 3-2 may include Reaction 3-2A or Reaction 3-2B.

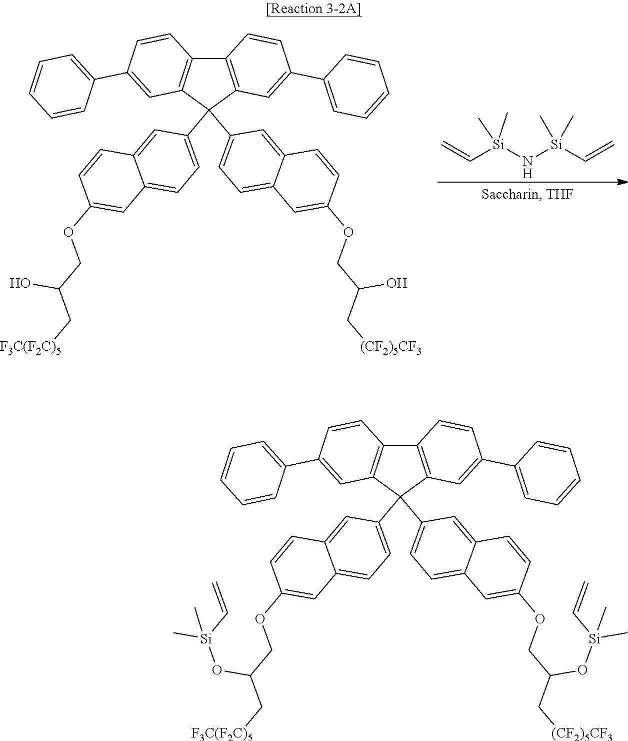

By Reaction 3-2A, the resist compound represented by Formula 5-3 may be produced.

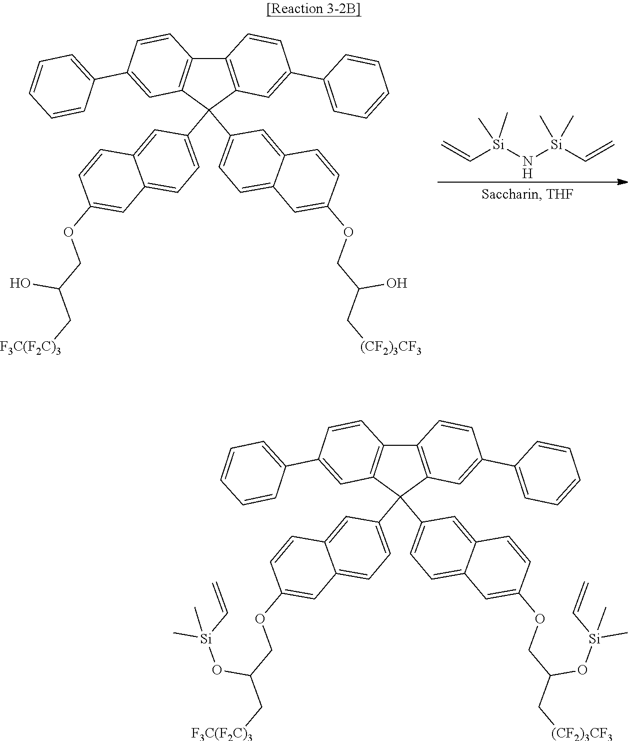

By Reaction 3-2B, the resist compound represented by Formula 5-4 may be produced.

### [Experimental Example 1-1] Synthesis of Resist Compound (HNF-OH) Represented by Formula 4-1 (Reaction 2-1A)

To a 100 cm³ round flask, 9,9-bis(6-hydroxy-2-naphthyl)fluorene (3.00 g, 6.66 mmol), 3-(perfluoro-n-hexyl)propenoxide (6.26 g, 16.7 mmol) and 4-dimethylaminopyridine (DMAP, 0.0200 g, 0.170 mmol) were injected, and a mixture was prepared using ethanol (30 cm³) as a solvent. The mixture was stirred at a temperature of about 90° C. for about 12 hours to produce a reaction solution. After finishing the reaction, the reaction solution was diluted using ethyl acetate (150 cm³). The organic solvent layer of the diluted reaction solution was separated and recovered, washed with water twice, and further washed once using a saturated sodium chloride aqueous solution (150 cm³). Then, moisture contained in the organic solvent layer was removed using anhydrous MgSO₄, and the organic solvent layer was concentrated under reduced pressure conditions to produce a product. The product was purified by column chromatography using a mixture of ethyl acetate and chloroform (ethyl acetate:chloroform=1:9) as a mobile phase, and then concentrated under a reduced pressure. The concentrated product was diluted again in ethyl acetate (20 cm³) and purified by re-precipitating in hexane (350 cm³) to obtain a final product (white solid, HNF-OH, 2.00 g, yield 25%).

### [Nuclear Magnetic Resonance (NMR) Analysis Results]

¹H NMR (400 MHZ, CDCl₃): δ=7.83 (d, J=7.5 Hz, 2H, Ar—H), 7.63 (d, J=9.0 Hz, 2H, Ar—H), 7.59-7.53 (m, 4H, Ar—H), 7.50 (d, J=7.5 Hz, 2H, Ar—H), 7.44-7.36 (m, 4H, Ar—H), 7.29 (td, J=7.5, 1.0 Hz, 2H, Ar—H), 7.12-7.07 (m, 4H, Ar—H), 4.61-4.52 (m, 2H, —CH(OH)—), 4.21-4.07 (m, 4H, —OCH₂—), 2.58-2.41 (m, 6H, —CH₂CF₂—, —OH); (ESI-TOF-MS): m/z Calc for (M+CHOO)⁻, 1225.1784; found, 1225.1792 (Here, Ar means a benzene ring).

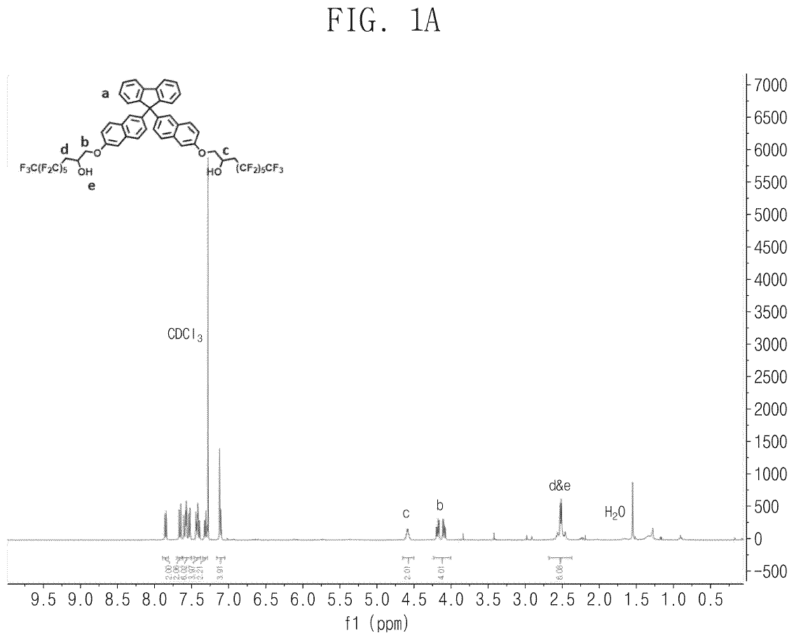

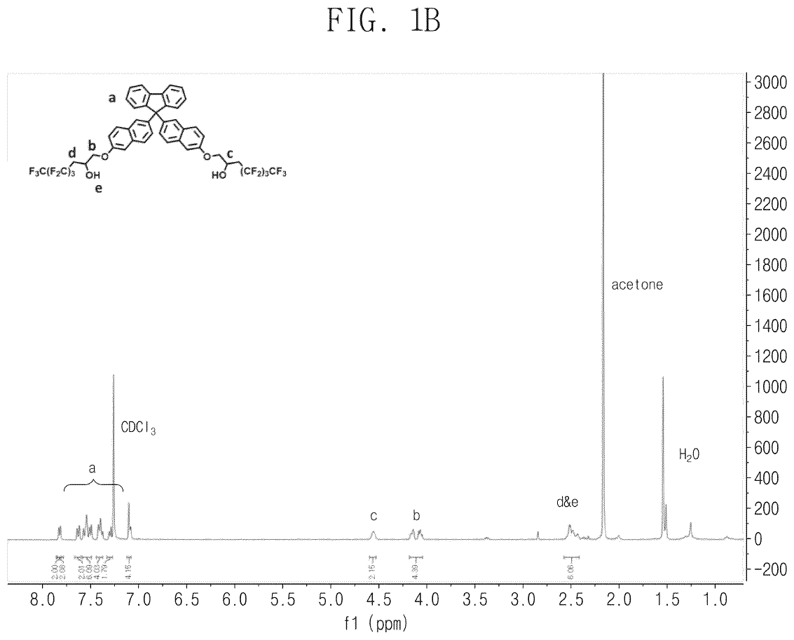

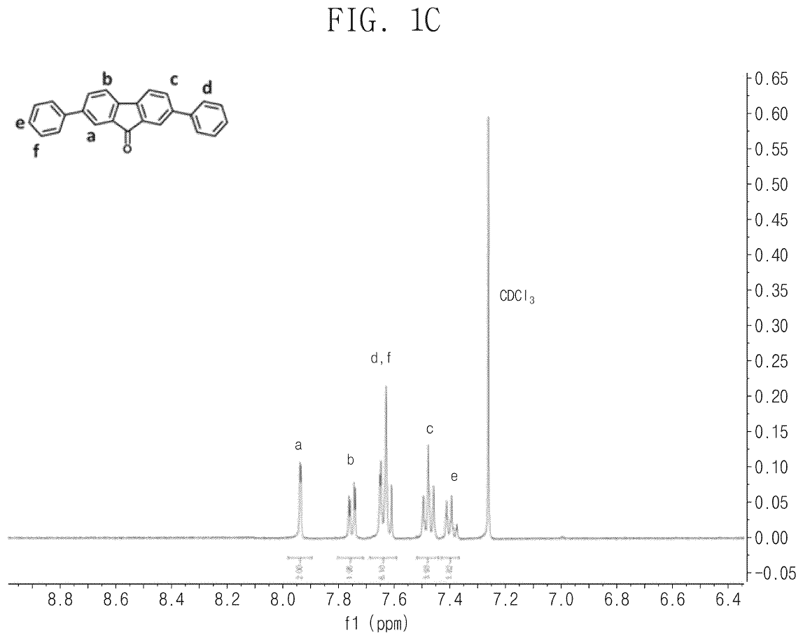

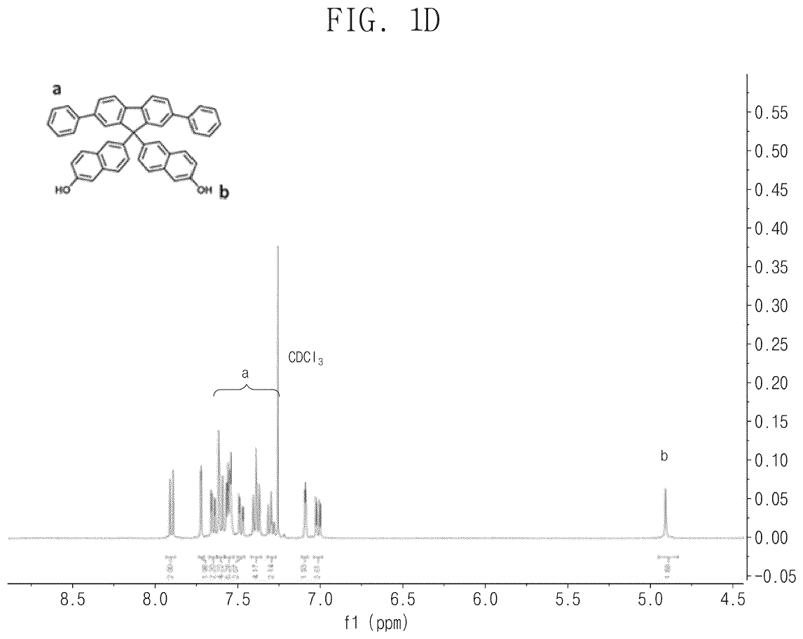

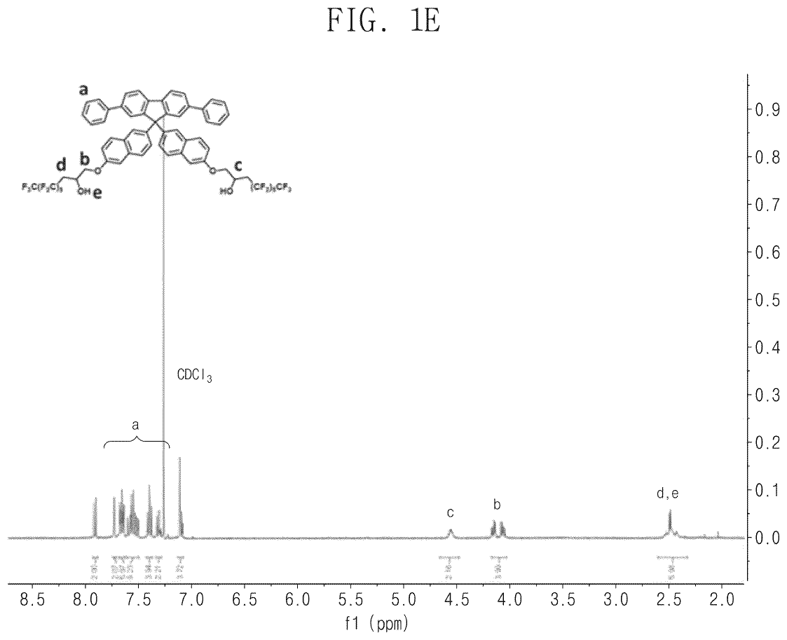

FIG. 1A is a graph showing the nuclear magnetic resonance spectrum results of a resist compound (HNF-OH) synthesized according to Experimental Example 1-1. Referring to FIG. 1A, the resist compound synthesized according to Experimental Example 1-1 was confirmed to have the structure of Formula 4-1.

### [Experimental Example 1-2] Synthesis of Resist Compound (HNF-OH(s)) Represented by Formula 4-2 (Reaction 2-1B)

To a 50 cm³ seal tube, 9,9-bis(6-hydroxy-2-naphthyl)fluorene (0.760 g, 1.69 mmol), 3-(perfluoro-n-butyl)propenoxide (1.08 g, 3.72 mmol) and 4-dimethylaminopyridine (DMAP, 0.0051 g, 0.0422 mmol) were injected, and a mixture was prepared using ethanol (7 cm³) as a solvent. The mixture was stirred at a temperature of about 90° ° C. for about 12 hours to produce a reaction solution. After finishing the reaction, the reaction solution was diluted using ethyl acetate (100 cm³). The organic solvent layer of the diluted reaction solution was separated and recovered, washed with water twice, and further washed once using a saturated sodium chloride aqueous solution (100 cm³). Then, moisture contained in the organic solvent layer was removed using anhydrous MgSO₄, and the organic solvent layer was concentrated under reduced pressure conditions to produce a product. The product was purified by column chromatography using a mixture of ethyl acetate and chloroform (ethyl acetate:chloroform=1:15) as a mobile phase. The product was concentrated under a reduced pressure to obtain a final product (white solid, HNF-OH(s), 0.66 g, yield 39%).

### [Nuclear Magnetic Resonance (NMR) Analysis Results]

¹H NMR (400 MHZ, CDCl₃): δ=7.82 (d, J=7.0 Hz, 2H, Ar—H), 7.63 (d, J=9.0 Hz, 2H, Ar—H), 7.60-7.52 (m, 4H, Ar—H), 7.50 (d, J=7.5 Hz, 2H, Ar—H), 7.40 (t, J=9.0 Hz, 4H, Ar—H), 7.30 (d, J=7.0 Hz, 2H, Ar—H), 7.09 (d, J=8.0 Hz, 4H, Ar—H), 4.60-4.52 (m, 2H, —CH(OH)—), 4.20-4.03 (m, 4H, —OCH₂—), 2.57-2.40 (m, 6H, —CH₂CF₂—, —OH) (Here, Ar means a benzene ring).

FIG. 1B is a graph showing the nuclear magnetic resonance spectrum results of a resist compound (HNF-OH(s)) synthesized according to Experimental Example 1-2. Referring to FIG. 1B, the resist compound synthesized according to Experimental Example 1-2 was confirmed to have the structure of Formula 4-2.

### [Experimental Example 1-3] Synthesis of Single Molecule (DPNF) Represented by Formula 6-2

### 1) Synthesis of diphenyl-9H-fluoren-9-one (DPF) (Reaction 4)

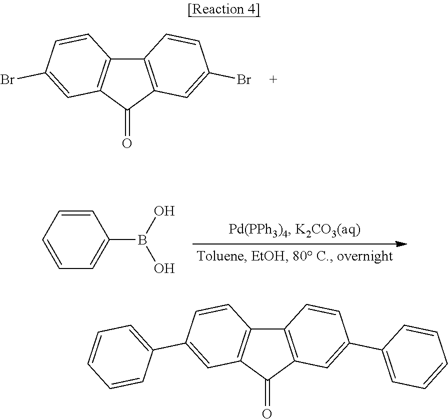 [SMILES: O=C1c2cc(Br)ccc2-c2ccc(Br)cc21 ; OB(O)c1ccccc1 ; O=C1c2cc(-c3ccccc3)ccc2-c2ccc(-c3ccccc3)cc21]

To a 250 cm³ Schlenk tube, 2,7-dibromo-9-fluorenone (2.40 g, 7.10 mmol), phenylboronic acid (2.16 g, 17.8 mmol), and tetrakis(triphenylphosphine)palladium(0) (0.0821 g, 0.0710 mmol) were injected and sealed. The inside of the tube was changed to a N₂ atmosphere using a vacuum pump. Toluene (80 cm³) and ethanol (20 cm³) were bubbled and injected into the tube, and a 2 M potassium carbonate aqueous solution (2.21 g, 16.0 mmol) was injected into the tube to produce a reaction solution. The reaction solution was stirred at a temperature of about 80ºC for about 12 hours. After finishing the reaction, the solvent of the reaction solution was vaporized using a rotary evaporator, and a preliminary product was obtained. The preliminary product was diluted using dichloromethane (250 cm³), and the organic solvent layer of the diluted preliminary product was washed with water twice and washed once more using a saturated sodium chloride aqueous solution (250 cm³). Then, moisture contained in the organic solvent layer was removed using anhydrous MgSO₄, and the moisture-removed organic solvent layer was concentrated under reduced pressure conditions to produce a product. The product was purified by column chromatography using chloroform as a mobile phase, and the product was concentrated under a reduced pressure. The concentrated product was purified through recrystallization using dichloromethane to obtain a final product (yellow solid, 2,7-diphenyl-9H-fluoren-9-one (DPF), 1.90 g, yield 80%).

### [Nuclear Magnetic Resonance (NMR) Analysis Results]

¹H NMR (400 MHZ, CDCl₃): δ=7.94 (dd, J=2.0, 0.5 Hz, 2H, Ar—H), 7.75 (dd, J=8.0, 2.0 Hz, 2H, Ar—H), 7.66-7.59 (m, 6H, Ar—H), 7.51-7.44 (m, 4H, Ar—H), 7.42-7.36 (m, 2H, Ar—H)

### 2) Synthesis of 2,7-diphenyl-9,9-bis(6-hydroxy-2-naphthyl)fluorene (DPNF) (Reaction 5)

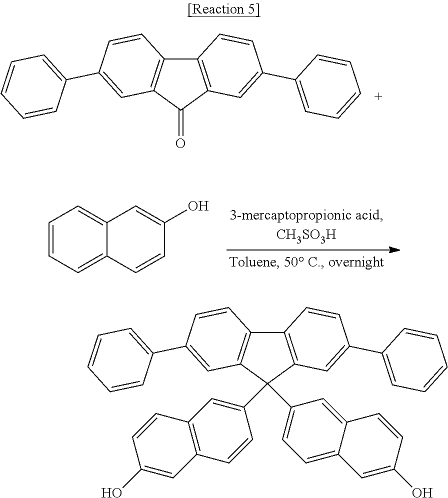 [SMILES: O=C1c2cc(-c3ccccc3)ccc2-c2ccc(-c3ccccc3)cc21 ; Oc1ccc2ccccc2c1 ; Oc1ccc2cc(C3(c4ccc5cc(O)ccc5c4)c4cc(-c5ccccc5)ccc4-c4ccc(-c5ccccc5)cc43)ccc2c1]

To a 100 cm³ round flask, 2,7-diphenyl-9H-fluoren-9-one (DPF, 1.40 g, 4.21 mmol), 2-naphthol (1.46 g, 10.1 mmol), 3-mercaptopropionic acid (0.0447 g, 0.421 mmol) and methanesulfonic acid (1.62 g, 16.8 mmol) were injected, and a reaction solution was produced using toluene (8.4 cm³) as a solvent. The reaction solution was stirred at a temperature of about 50° C. for about 12 hours. The reaction solution was neutralized using a 2 M NaOH aqueous solution and diluted using ethyl acetate (100 cm³). The organic solvent layer of the diluted reaction solution was washed with water twice and washed once more using a saturated sodium chloride aqueous solution (100 cm³). Then, moisture contained in the organic solvent layer was removed using anhydrous MgSO₄, and the moisture-removed organic solvent layer was concentrated under reduced pressure conditions to produce a product. The product was purified by column chromatography using a mixture of dichloromethane and ethyl acetate (dichloromethane:ethyl acetate=15:1) as a mobile phase, and the product was concentrated under a reduced pressure. As a result, a final product (brown solid, 2,7-diphenyl-9,9-bis(6-hydroxy-2-naphthyl)fluorene (DPNF), 0.9 g, yield 35%) was obtained.

### [Nuclear Magnetic Resonance (NMR) Analysis Results]

¹H NMR (400 MHZ, CDCl₃): δ=7.90 (d, J=8.0 Hz, 2H, Ar—H), 7.73 (d, J=1.0 Hz, 2H, Ar—H), 7.65 (dd, J=8.0, 1.5 Hz, 2H, Ar—H), 7.61 (d, J=9.0 Hz, 4H, Ar—H), 7.59-5.52 (m, 6H, Ar—H), 7.48 (dd, J=9.0, 2.0 Hz, 2H, Ar—H), 7.42-7.36 (m, 4H, Ar—H), 7.33-7.27 (m, 2H, Ar—H), 7.10 (d, J=2.5 Hz, 2H, Ar—H), 7.02 (dd, J=9.0, 2.5 Hz, 2H, Ar—H), 4.91 (s, 2H, —OH) (Here, Ar means a benzene ring).

FIG. 1C and FIG. 1D are graphs showing the nuclear magnetic resonance spectrum results of DPF and DPNF, synthesized according to Experimental Example 1-3. Referring to FIG. 1C and FIG. 1D, the final synthesis of the single molecule (DPNF) of Formula 6-2 was confirmed according to Experimental Example 1-3.

### [Experimental Example 1-4] Synthesis of Resist Compound (DPNF-OH) Represented by Formula 4-3 (Reaction 2-2A)

To a 100 cm³ round flask, 2,7-diphenyl-9,9-bis(6-hydroxy-2-naphthyl)fluorene (DPNF, 0.770 g, 1.28 mmol), perfluoroalkyl epoxide (1.20 g, 3.19 mmol) and 4-dimethylaminopyridine (DMAP, 0.00390 g, 0.0319 mmol) were injected, and a mixture was prepared using ethanol (10 cm³) as a solvent. The mixture was stirred at a temperature of about 90° ° C. for about 12 hours to produce a reaction solution. After finishing the reaction, the reaction solution was diluted using ethyl acetate (100 cm³). The organic solvent layer of the diluted reaction solution was separated and recovered, washed with water twice, and further washed once using a saturated sodium chloride aqueous solution (100 cm³). Then, moisture contained in the organic solvent layer was removed using anhydrous MgSO₄, and the organic solvent layer was concentrated under reduced pressure conditions to produce a product. The product was purified by column chromatography using a mixture of ethyl acetate and hexane (ethyl acetate:hexane=1:2) as a mobile phase, and then concentrated under a reduced pressure. The concentrated product was diluted again in ethyl acetate (3 cm³) and re-precipitated in hexane (200 cm³) to obtain a final product (white solid, DPNF-OH, 0.700 g, yield 40%).

### [Nuclear Magnetic Resonance (NMR) Analysis Results].

¹H NMR (400 MHz, CDCl₃): δ=7.91 (d, J=9.0 Hz, 2H, Ar—H), 7.73 (d, J=1.5 Hz, 2H, Ar—H), 7.67 (t, J=1.5 Hz, 2H, Ar—H), 7.65 (d, J=2.0 Hz, 2H, Ar—H), 7.64 (d, J=1.5 Hz, 2H, Ar—H), 7.61-7.53 (m, 6H, Ar—H), 7.51 (dd, J=9.0, 2.0 Hz, 2H, Ar—H), 7.42-7.37 (m, 4H, Ar—H), 7.34-7.28 (m, 2H, Ar—H), 7.12-7.08 (m, 4H, Ar—H), 4.60-4.51 (m, 2H, —CH(OH)—), 4.18-4.04 (m, 4H, —OCH₂—), 2.57-2.41 (m, 6H, —CH₂CF₂—, —OH) (Here, Ar means a benzene ring).

FIG. 1E is a graph showing the nuclear magnetic resonance spectrum results of a resist compound (DPNF-OH) synthesized according to Experimental Example 1-4. Referring to FIG. 1E, the resist compound synthesized according to Experimental Example 1-4 was confirmed to have the structure of Formula 4-3.

### [Experimental Example 2-1] Synthesis of Resist Compound (HNF-DVS) Represented by Formula 5-1 (Reaction 3-1A)

To a 50 cm³ seal tube, HNF-OH (1.00 g, 0.830 mmol), 1,3-divinyl-1,1,3,3-tetramethyldisilazane (0.340 g, 1.83 mmol) and saccharin (1.70 mg, 0.00910 mmol) were injected, and a mixture was prepared using tetrahydrofuran (THF, 20 cm³) as a solvent. The mixture was stirred at a temperature of about 60° ° C. for about 2 hours to produce a reaction solution. After finishing the reaction, the reaction solution was diluted using ethyl acetate (100 cm³). The organic solvent layer of the diluted reaction solution was washed with water twice, and further washed once using a saturated sodium chloride aqueous solution (100 cm³). Then, moisture contained in the organic solvent layer was removed using anhydrous MgSO₄, and the organic solvent layer was concentrated under reduced pressure conditions to produce a product. The product was purified by column chromatography using a mixture of ethyl acetate and hexane (ethyl acetate:hexane=1:9) as a mobile phase. The product was concentrated under reduced pressure conditions to obtain a final product (white solid, HNF-DVS, 0.800 g, yield 71%).

### [Nuclear Magnetic Resonance (NMR) Analysis Results]

¹H NMR (400 MHZ, CDCl₃): δ=7.83 (d, J=7.0 Hz, 2H, Ar—H), 7.63 (d, J=9.0 Hz, 2H, Ar—H), 7.55 (d, J=9.0 Hz, 4H, Ar—H), 7.51 (d, J=7.5 Hz, 2H, Ar—H), 7.40 (t, J=7.5 Hz, 4H, Ar—H), 7.29 (t, J=7.5 Hz, 2H, Ar—H), 7.12-7.02 (m, 4H, Ar—H), 6.18 (dd, J=20.0, 15.0 Hz, 2H, —CHCH₂), 6.04 (dd, J=20.0, 3.5 Hz, 2H, —CHCH₂), 5.82 (dd, J=20.0, 3.5 Hz, 2H, —SiCH—), 4.61-4.52 (m, 2H, —CH(OSi—)—), 4.07-3.93 (m, 4H, —OCH₂—), 2.61-2.30 (m, 4H, —CH₂CF₂—), 0.25 (s, 12H, —CH₃); (ESI-TOF-MS): m/z Calc for (M+CHOO)⁻, 1415.2643; found, 1415.2682 (Here, Ar means a benzene ring).

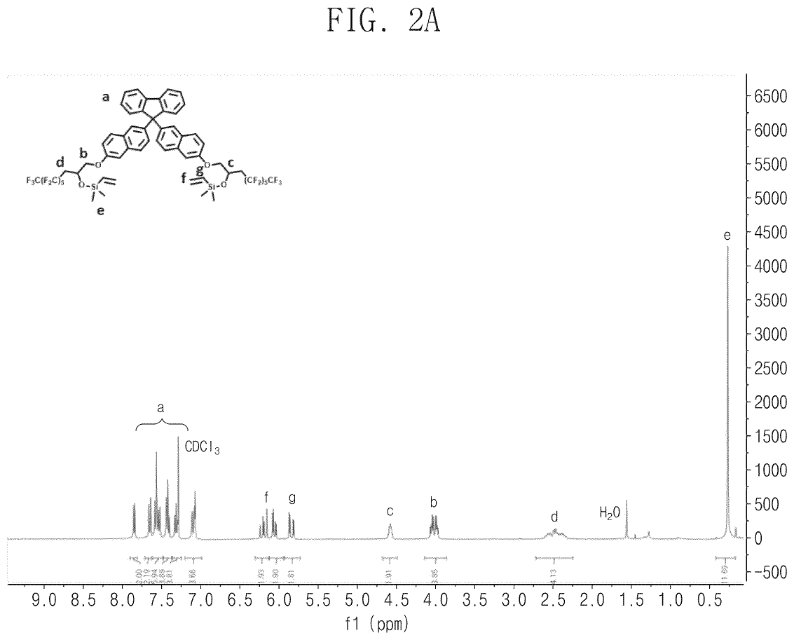

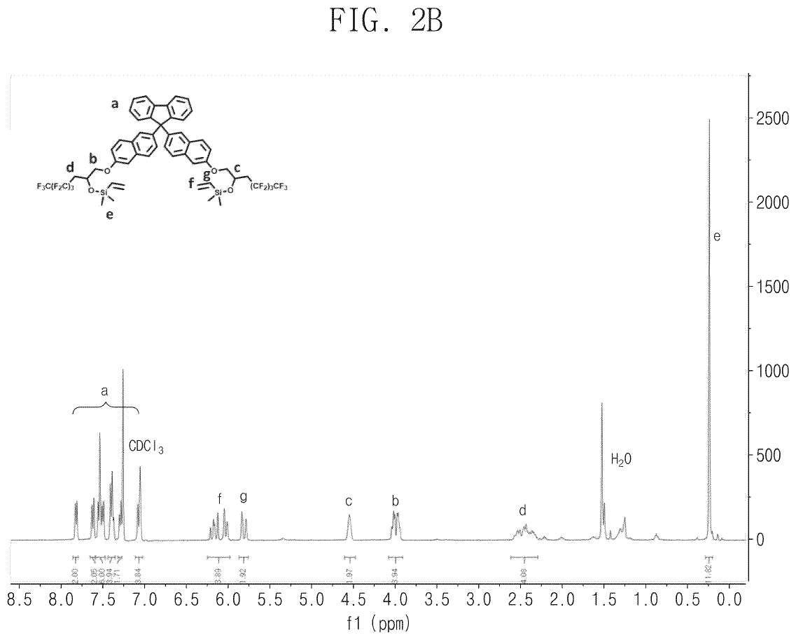

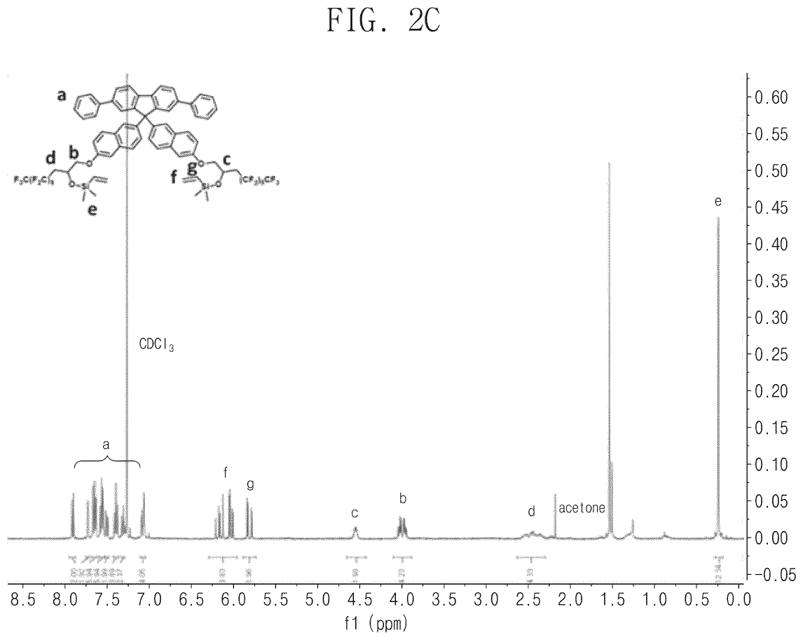

FIG. 2A is a graph showing the nuclear magnetic resonance spectrum results of a resist compound (HNF-DVS) synthesized according to Experimental Example 2-1. Referring to FIG. 2A, the resist compound synthesized according to Experimental Example 2-1 was confirmed to have a vinyl silyl group.

### [Experimental Example 2-2] Synthesis of Resist Compound (HNF-DVS(s)) Represented by Formula 5-2 (Reaction 3-1B)

To a 50 cm³ seal tube, HNF-OH(s) (0.80 g, 0.798 mmol), 1,3-divinyl-1,1,3,3-tetramethyldisilazane (0.325 g, 1.83 mmol) and saccharin (1.60 mg, 0.00878 mmol) were injected, and a mixture was prepared using tetrahydrofuran (THF, 10 cm³) as a solvent. The mixture was stirred at a temperature of about 60° C. for about 2 hours to produce a reaction solution. After finishing the reaction, the reaction solution was diluted using ethyl acetate (100 cm³). The organic solvent layer of the diluted reaction solution was washed with water twice, and further washed once using a saturated sodium chloride aqueous solution (100 cm³). Then, moisture contained in the organic solvent layer was removed using anhydrous MgSO₄, and the organic solvent layer was concentrated under reduced pressure conditions to produce a product. The product was purified by column chromatography using a mixture of ethyl acetate and hexane (ethyl acetate:hexane=1:9) as a mobile phase. The product was concentrated under a reduced pressure to obtain a final product (white solid, HNF-DVS(s), 0.700 g, yield 75%).

### [Nuclear Magnetic Resonance (NMR) Analysis Results]

¹H NMR (400 MHZ, CDCl₃): δ=7.82 (d, J=7.0 Hz, 2H, Ar—H), 7.62 (d, J=9.0 Hz, 2H, Ar—H), 7.55 (d, J=9.0 Hz, 4H, Ar—H), 7.50 (d, J=7.5 Hz, 2H, Ar—H), 7.39 (t, J=8.0 Hz, 4H, Ar—H), 7.29 (d, J=7.5 Hz, 2H, Ar—H), 7.07 (d, J=11.5 Hz, 4H, Ar—H), 6.18 (dd, J=19.5, 15.0 Hz, 2H, —CHCH₂), 6.03 (d, J=13.0 Hz, 2H, —CHCH₂), 5.82 (d, J=20.5 Hz, 2H, —SiCH—), 4.60-4.51 (m, 2H, —CH(OSi—)—), 4.06-3.92 (m, 4H, —OCH₂—), 2.60-2.31 (m, 4H, —CH₂CF₂—), 0.25 (s, 12H, —CH₃) (Here, Ar means a benzene ring).

FIG. 2B is a graph showing the nuclear magnetic resonance spectrum results of a resist compound (HNF-DVS(s)) synthesized according to Experimental Example 2-2. Referring to FIG. 2B, the resist compound synthesized according to Experimental Example 2-2 was confirmed to have a vinyl silyl group.

### [Experimental Example 2-3] Synthesis of Resist Compound (DPNF-DVS) Represented by Formula 5-3 (Reaction 3-2A)

To a 25 cm³ seal tube, DPNF-OH (0.60 g. 0.44 mmol), 1,3-divinyl-1,1,3,3-tetramethyldisilazane (0.16 g, 0.89 mmol) and saccharin (0.80 mg, 0.0044 mmol) were injected, and a mixture was prepared using tetrahydrofuran (THF, 6 cm³) as a solvent. The mixture was stirred at a temperature of about 60° C. for about 2 hours to produce a reaction solution. After finishing the reaction, the reaction solution was diluted using ethyl acetate (100 cm³). The organic solvent layer of the diluted reaction solution was washed with water twice, and further washed once using a saturated sodium chloride aqueous solution (100 cm³). Then, moisture contained in the organic solvent layer was removed using anhydrous MgSO₄, and the organic solvent layer was concentrated under reduced pressure conditions to produce a product. The product was purified by column chromatography using a mixture of chloroform and hexane (chloroform:hexane=3:1) as a mobile phase, and the product was concentrated under a reduced pressure. The concentrated product was diluted again in ethyl acetate (3 cm³) and purified through re-precipitation using methanol (150 cm³). As a result, a final product (white solid, DPNF-DVS, 0.42 g, yield 62%) was obtained.

### [Nuclear Magnetic Resonance (NMR) Analysis Results]

¹H NMR (400 MHZ, CDCl₃): δ=7.90 (d, J=8.0 Hz, 2H, Ar—H), 7.73 (d, J=1.5 Hz, 2H, Ar—H), 7.67 (d, J=1.0 Hz, 2H, Ar—H), 7.65 (d, J=1.5 Hz, 2H, Ar—H), 7.63 (d, J=1.5 Hz, 2H, Ar—H), 7.59-7.53 (m, 6H, Ar—H), 7.50 (dd, J=9.0, 2.0 Hz, 2H, Ar—H), 7.39 (t, J=7.5 Hz, 4H, Ar—H), 7.33-7.27 (m, 2H, Ar—H), 7.10-7.04 (m, 4H, Ar—H), 6.17 (dd, J=20.0, 15.0 Hz, 2H, —CHCH₂), 6.03 (dd, J=15.0, 4.0 Hz, 2H, —CHCH₂), 5.81 (dd, J=20.0, 4.0 Hz, 2H, —SiCH—), 4.60-4.50 (m, 2H, —CH(OSi—)—), 4.06-3.93 (m, 4H, —OCH₂—), 2.60-2.29 (m, 4H, —CH₂CF₂—), 0.24 (d, J=2.0 Hz, 12H, —CH₃) (Here, Ar means a benzene ring).

FIG. 2C is a graph showing the nuclear magnetic resonance spectrum results of a resist compound (DPNF-DVS) synthesized according to Experimental Example 2-3. Referring to FIG. 2C, the resist compound synthesized according to Experimental Example 2-3 was confirmed to have a vinyl silyl group.

### A Method for Manufacturing Semiconductor Device Using the Resist Compound According to Embodiments of the Inventive Concept Will be Explained.

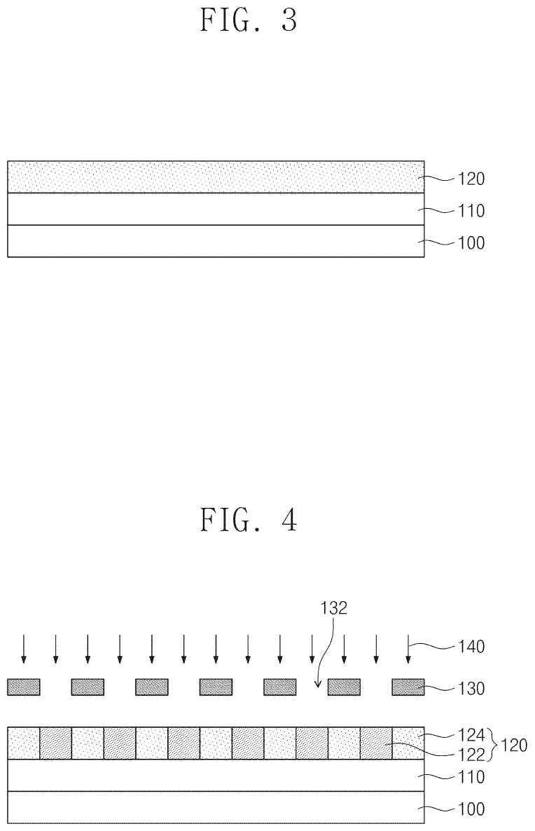

FIG. 3 to FIG. 6 are cross-sectional views showing a method for manufacturing a semiconductor device using the resist compound according to embodiments of the inventive concept.

Referring to FIG. 3, a lower layer 110 may be formed on a substrate 100, and on the lower layer 110, a photoresist layer 120 may be formed. The substrate 100 may be a semiconductor substrate, for example, a silicon substrate, a germanium substrate or a silicon/germanium substrate. The lower layer 110 may be an etching target layer and may be formed by any one selected among a semiconductor material, a conductive material, an insulating material, or combinations thereof. The lower layer 110 may be formed as a single layer or may include multiple layers stacked on the substrate 100.

The photoresist layer 120 may include the resist compound according to embodiments of the inventive concept. The resist compound may be represented by Formula 1. The formation of the photoresist layer 120 may include applying the resist compound on the lower layer 110 using a fluorine-based solvent. The fluorine-based solvent may include, for example, hydrofluoroether (HFE) and/or perfluorocarbon (PFC). The applying of the resist compound on the lower layer 110 may be performed by, for example, a spin coating method. The formation of the photoresist layer 120 may further include performing heating process (for example, a soft baking process) on the resist compound applied.

Referring to FIG. 4, an exposing process may be performed on the photoresist layer 120. The exposing process may include arranging a photomask 130 above the photoresist layer 120, and irradiating light 140 on the photoresist layer 120 through the photomask 130. The light 140 may be e-beam or extreme ultraviolet. The photoresist layer 120 may include a first part 122 exposed to the light 140 and a second part 124 unexposed to the light 140. The light 140 may be irradiated to the first part 122 through openings 132 of the photomask 130, and may not be irradiated to the second part 124 due to the block by the photomask 130.

The resist compound may include radicals produced by the irradiation of the light 140. A C—F bond in the resist compound may be broken by secondary electrons produced by the irradiation of the light 140, and accordingly, carbon radicals may be formed. The carbon radicals may be chemically combined with the vinyl silyl group of the resist compound. In an embodiment, double bonds between the carbon atoms of the vinyl silyl group may be broken, and the vinyl silyl group and the carbon radicals may be combined. Through the combination reaction of the vinyl silyl group and the carbon radicals, the resist compound may include a combined (crosslinked) structure of the molecules represented by Formula 1 from each other.

In the first part 122 of the photoresist layer 120, the resist compound may include the carbon radicals produced by the irradiation of the light 140. Through the combination reaction of the vinyl silyl group and the carbon radicals, the resist compound may include a combined (crosslinked) structure of the molecules represented by Formula 1 from each other. In the second part 124 of the photoresist layer 120, the chemical structure of the resist compound may not change, and the resist compound may include a single molecular structure represented by Formula 1. By the exposing process, the first part 122 and the second part 124 of the photoresist layer 120 may have different chemical structures from each other.

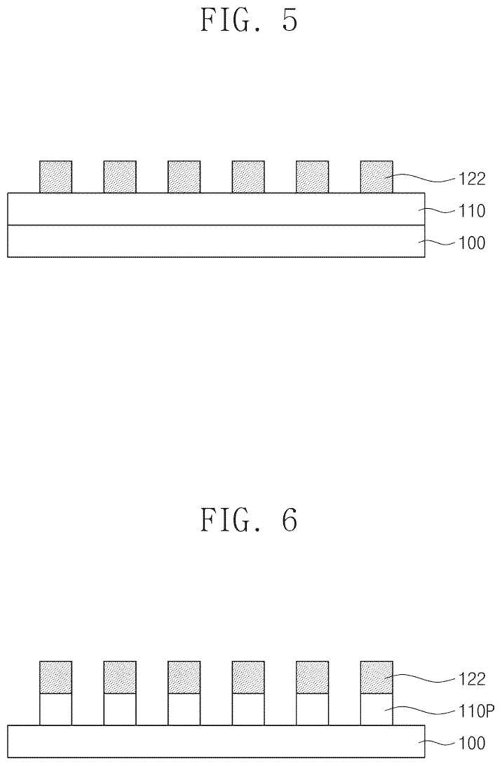

Referring to FIG. 5, after the exposing process, the photomask 130 may be removed. A developing process may be performed on the exposed photoresist layer 120. The developing process may include removing the second part 124 of the photoresist layer 120 by using a developing solution. The developing solution may include a fluorine-based solvent and a solution including thereof. The fluorine-based solvent may include, for example, hydrofluoroether (HFE) and/or perfluorocarbon (PFC). By the developing process, the second part 124 of the photoresist layer 120 may be selectively removed. The first part 122 of the photoresist layer 120 may be referred to as a photoresist pattern, and the photoresist pattern 122 may be a negative tone pattern.

According to embodiments of the inventive concept, the resist compound may have an organic single molecular structure represented by Formula 1. Since the resist compound has the organic single molecular structure having a small molecular weight in contrast to a polymer, the resist compound may be purified so as to have high purity through a sublimation process under a low pressure.

The photoresist layer 120 may include the resist compound of the organic single molecular structure represented by Formula 1. By the exposing process, the resist compound in the exposed part (i.e., the first part 122) of the photoresist layer 120 may include a combined (or crosslinked) structure of the molecules represented by Formula 1 from each other, and the resist compound in the unexposed part (i.e., the second part 124) of the photoresist layer 120 may include the single molecular structure represented by Formula 1.

Since the resist compound includes a fluorine atom having high absorbance of the light 140 (for example, extreme ultraviolet), the production of the carbon radicals in the first part 122 of the photoresist layer 120 may increase. Further, the resist compound may include a vinyl silyl group which may undergo the combination reaction with the carbon radicals, and accordingly, the crosslinking bond between the molecules represented by Formula 1 in the first part 122 of the photoresist layer 120 may be easy. In the second part 124 of the photoresist layer 120, the resist compound may have a single molecular structure having a small size (or molecular weight) and narrow polydispersity index in contrast to a polymer, and accordingly, the second part 124 may have a high and uniform solubility with respect to the developing solution used in the developing process. Accordingly, the sensitivity and resolution of the photoresist pattern 122 having a negative tone may be improved.

Further, the developing process may be performed using the fluorine-based solvent as a developing solution. The fluorine-based solvent has no flammability, low toxicity, chemical stability and relatively low surface tension. Since the developing process is performed using the fluorine-based solvent having relatively low surface tension, the pattern collapse of the photoresist pattern 122 may be minimized.

Referring to FIG. 6, the lower layer 110 may be etched using the photoresist pattern 122 as an etching mask. The etching of the lower layer 110 may include, for example, a wet or dry etching process. The lower layer 110 may be etched to form a lower pattern 110P. After forming the lower pattern 110P, the photoresist pattern 122 may be removed. The lower pattern 110P may be a semiconductor pattern, a conductive pattern, or an insulating pattern in a semiconductor device.

According to embodiments of the inventive concept, the resist compound may have an organic single molecular structure represented by Formula 1 and may have high etching resistance due to the core structure constituting the organic single molecular structure. Accordingly, during the etching process of the lower layer 110, the etching resistance of the photoresist pattern 122 may increase, and thus, the formation of the lower pattern 110P may be easy. Further, the resist compound may not include a metal element, and accordingly, the contamination by the metal element may be prevented during the manufacturing process of a semiconductor device.

According to the inventive concept, the resist compound may have an organic single molecular structure represented by Formula 1, and the photoresist layer 120 may include the resist compound. By performing the exposing process and the developing process on the photoresist layer 120, the photoresist pattern 122 may be formed. Since the resist compound has the organic single molecular structure represented by Formula 1, the sensitivity and resolution of the photoresist pattern 122 may be improved, and the etching resistance of the photoresist pattern 122 may be increased. Further, the developing process may be performed using the fluorine-based solvent, and accordingly, the pattern collapse of the photoresist pattern 122 may be minimized.

Accordingly, a resist compound which may improve the resolution and sensitivity of a photoresist pattern, increase the etching resistance of the photoresist pattern, and may restrain the collapse of the photoresist pattern, a method for forming the same and a method for manufacturing a semiconductor device using the same may be provided.

### [Experimental Example 3] Formation of Photoresist Pattern by e-Beam Lithography Process

A HNF-OH solution (about 3 wt/vol %) in which the resist compound (HNF-OH) synthesized in Experimental Example 1-1 was dissolved in PF-7600 (3M Co.) was applied on a silicon substrate at about 3000 rpm for about 60 seconds by a spin coating method, and heated at about 80° C. for about 1 minute to form a first photoresist thin layer (a thickness of about 100 nm). On the photoresist thin layer, e-beam of about 50 to about 1,500 μC/cm² was irradiated under an acceleration voltage of about 80 keV, and then, a developing process was performed using a fluorine-based developing solution (HFE-7300 (3M Co.): PF-7600 (3M Co.)=1:1) for about 35 seconds to form a negative tone first photoresist pattern.

A HNF-DVS solution (about 2.2 wt/vol %) in which the resist compound (HNF-DVS) synthesized in Experimental Example 2-1 was dissolved in PF-7600 (3M Co.) was applied on a silicon substrate at about 3000 rpm for about 60 seconds by a spin coating method, and heated at about 80° C. for about 1 minute to form a second photoresist thin layer (a thickness of about 100 nm). On the second photoresist thin layer, e-beam of about 50 to about 1,500 μC/cm² was irradiated under an acceleration voltage of about 80 keV, and then, a developing process was performed using a fluorine-based developing solution (HFE-7300: FC-3283(3M Co.)=1:1) for about 35 seconds to form a negative tone second photoresist pattern.

A HNF-DVS(s) solution (about 2.3 wt/vol %) in which the resist compound (HNF-DVS(s)) synthesized in Experimental Example 2-2 was dissolved in PF-7600 (3M Co.) was applied on a silicon substrate at about 4000 rpm for about 60 seconds by a spin coating method, and heated at about 80° ° C. for about 1 minute to form a third photoresist thin layer (a thickness of about 90 nm). On the third photoresist thin layer, e-beam of about 50 to about 1,500 μC/cm² was irradiated under an acceleration voltage of about 80 keV, and then, a developing process was performed using a fluorine-based developing solution (HFE-7300) for about 30 seconds to form a negative tone third photoresist pattern.

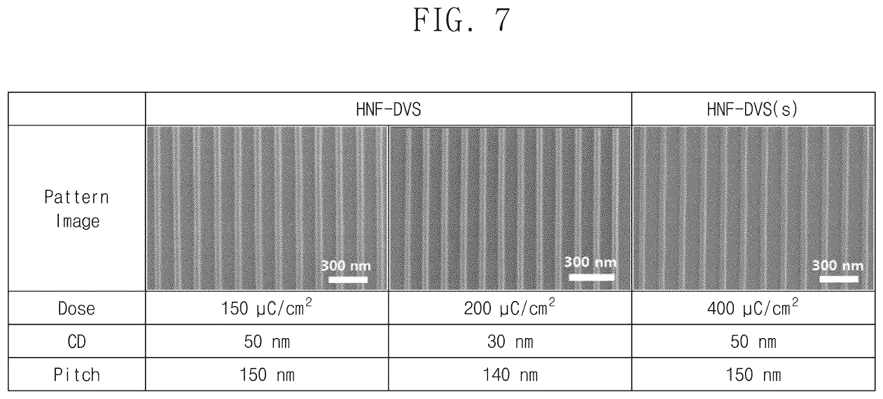

FIG. 7 shows scanning electron microscopic images of the photoresist patterns formed according to Experimental Example 3. Referring to FIG. 7, in the case of the resist compound (HNF-DVS) represented by Formula 5-1, during performing a developing process using a fluorine-based developing solution (HFE-7300: FC-3283=1:1), if e-beam of about 150 μC/cm² was irradiated, a negative tone photoresist pattern with a line with of about 50 nm was formed, and if e-beam of about 200 μC/cm² was irradiated, a negative tone photoresist pattern with a line width of about 30 nm was formed. Further, in the case of the resist compound (HNF-DVS(s)) represented by Formula 5-2, during performing a developing process using a fluorine-based developing solution (HFE-7300), if e-beam of about 400 μC/cm² was irradiated, a negative tone photoresist pattern with a line with of about 50 nm was formed. That is, in the cases of the resist compounds (HNF-DVS and HNF-DVS(s)), it could be confirmed that negative tone photoresist patterns having excellent sensitivity to the e-beam can be formed.

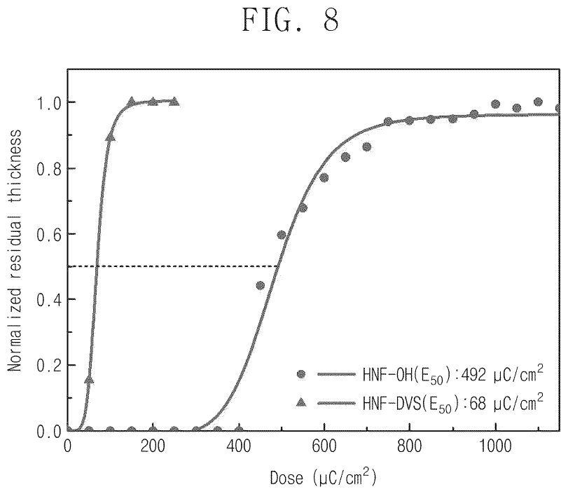

FIG. 8 is a graph showing the solubility evaluation results of a photoresist thin layer formed according to Experimental Example 3. Referring to FIG. 8, when measuring dose to gel (dosage at the point of curing the photoresist thin layer) through e-beam lithography, in the case of the resist compound (HNF-OH) represented by Formula 4-1, if e-beam of about 492 μC/cm² was irradiated, the thickness of the first photoresist pattern could be maintained to about 50% of the thickness of the first photoresist thin layer. In the case of the resist compound (HNF-DVS) represented by Formula 5-1, if e-beam of about 68 μC/cm² was irradiated, the thickness of the second photoresist pattern could be maintained to about 50% of the thickness of the second photoresist thin layer. That is, it could be confirmed that a negative tone photoresist pattern having excellent sensitivity to the e-beam can be formed by using the resist compound according to embodiments of the inventive concept.

### [Experimental Example 4] Evaluation of Solubility Change of Photoresist Thin Layer with Respect to Extreme Ultraviolet

A HNF-DVS solution (about 2.5 wt/vol %) obtained by dissolving the resist compound (HNF-DVS) synthesized in Experimental Example 2-1 in PF-7600 (3M Co.) was applied on a silicon substrate at about 3000 rpm for about 60 seconds by a spin coating method, and heated at about 80ºC for about 1 minute to form a photoresist thin layer (a thickness of about 120 nm). Extreme ultraviolet of about 2 to about 45 mJ/cm² was irradiated on the photoresist thin layer using a stepper installed in a 4A beam line of Pohang Accelerator Laboratory, and then, a developing process was performed using a fluorine-based developing solution (HFE-7300: FC-3283=1:1) for about 90 seconds to form a negative tone photoresist pattern. The thickness of the photoresist pattern was measured using Alpha-Step® D-300 stylus profiler manufactured by Kla-Tencor Co., and the solubility change properties of the photoresist thin layer were evaluated.

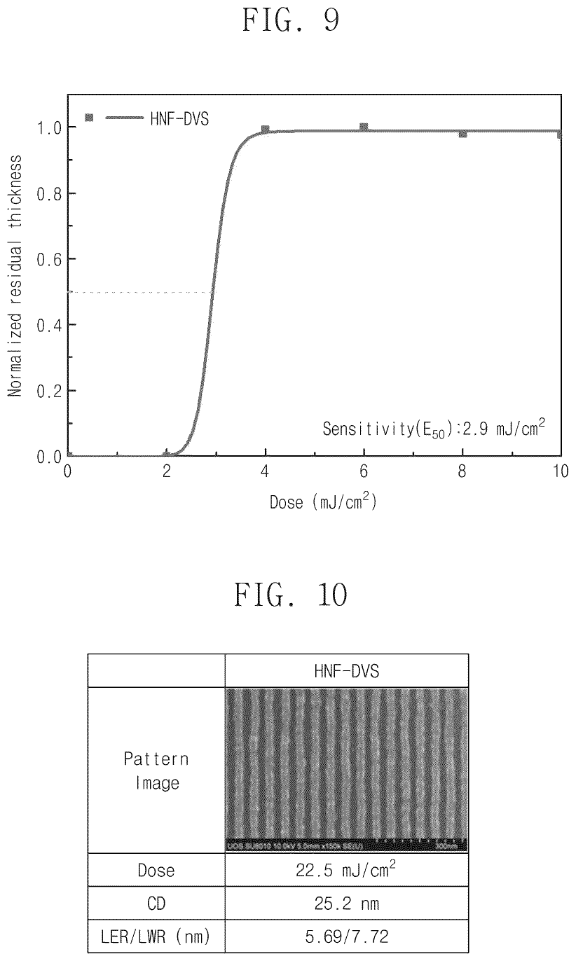

FIG. 9 is a graph showing the solubility evaluation results of a photoresist thin layer formed according to Experimental Example 4. Referring to FIG. 9, in the case of the resist compound (HNF-DVS) represented by Formula 5-1, if extreme ultraviolet of about 2.9 mJ/cm² was irradiated, the thickness of the photoresist pattern could be maintained to about 50% of the thickness of the photoresist thin layer. That is, it could be confirmed that a negative tone photoresist pattern having excellent sensitivity to the extreme ultraviolet can be formed by using the resist compound according to embodiments of the inventive concept.

### [Experimental Example 5] Formation of Photoresist Pattern by Extreme Ultraviolet Lithography Process

A HNF-DVS solution (about 0.85 wt/vol %) obtained by dissolving the resist compound (HNF-DVS) synthesized in Experimental Example 2-1 in PF-7600 was applied on a silicon substrate coated with 1,3-divinyl-1,1,3,3-tetramethyldisilazane (DVS) at about 805 rpm for about 60 seconds by a spin coating method, and heated at about 80° C. for about 1 minute to form a photoresist thin layer (a thickness of about 35 nm). Extreme ultraviolet (EUV) was irradiated on the photoresist thin layer using a MET5 stepper possessed by Lawrence Berkeley National Laboratory, and then, a developing process was performed using a fluorine-based developing solution (HFE-7300: FC-3283=2:3) for about 30 seconds to form a negative tone photoresist pattern having a line width of about 30 nm or less.

FIG. 10 shows a scanning electron microscope image of a photoresist pattern formed according to Experimental Example 5. Referring to FIG. 10, it could be confirmed that if extreme ultraviolet of about 22.5 mJ/cm² was irradiated, a negative tone photoresist pattern having a line width of about 30 nm or less (that is, a line width of about 25.2 nm) was formed. That is, in the case of the resist compound (HNF-DVS), it could be confirmed that a negative tone photoresist pattern having excellent sensitivity to the extreme ultraviolet can be formed.

### [Experimental Example 6] Evaluation of Etching Resistance of Photoresist Thin Layer

Experiments on comparing etching rates of the resist compound (HNF-OH) synthesized in Experimental Example 1-1, the resist compound (HNF-DVS) synthesized in Experimental Example 2-1, a resist (L6) disclosed in Korean Registration Patent No. 10-2215511, and a commercially available KrF resist were conducted.

1) A HNF-OH solution (about 10 wt/vol %) obtained by dissolving the resist compound (HNF-OH) synthesized in Experimental Example 1-1 in PF-7600 was applied on a silicon substrate at about 1500 rpm for about 60 seconds by a spin coating method, and heated at about 80° C. for about 1 minute to form a first photoresist thin layer (a thickness of about 610 nm).

2) A HNF-DVS solution (about 10 wt/vol %) obtained by dissolving the resist compound (HNF-DVS) synthesized in Experimental Example 2-1 in PF-7600 was applied on a silicon substrate at about 2000 rpm for about 60 seconds by a spin coating method, and heated at about 80° C. for about 1 minute to form a second photoresist thin layer (a thickness of about 500 nm).

3) (Comparative Example 1) A solution of L6 (about 20 wt/vol %) obtained by dissolving a resist (L6) disclosed in Korean Registration Patent No. 10-2215511 in HFE-7500 was applied on a silicon substrate at about 1000 rpm for about 60 seconds by a spin coating method, and heated at about 80° ° C. for about 1 minute to form a third photoresist thin layer (a thickness of about 1 μm).

4) (Comparative Example 2) A commercially available KrF resist solution (purchased from Dongjin Semichem) was applied on a silicon substrate at about 2000 rpm for about 60 seconds by a spin coating method, and heated at about 80ºC for about 1 minute to form a fourth photoresist thin layer (a thickness of about 630 nm).

5) An etching process was performed on the first to fourth photoresist thin layers. The etching process is a dry etching process using a reactive ion etching (RIE) method, and a mixture gas of O₂ and CF₄ was used as an etching gas. The etching process was performed by about 100 W, about 100 kHz, and flow conditions of about 2 sccm of O₂ and about 18 sccm CF₄. The thickness of a remaining thin film according to the etching time (0 seconds, 25 seconds, 50 seconds and 75 seconds) of the etching process was measured using ellipsometry.

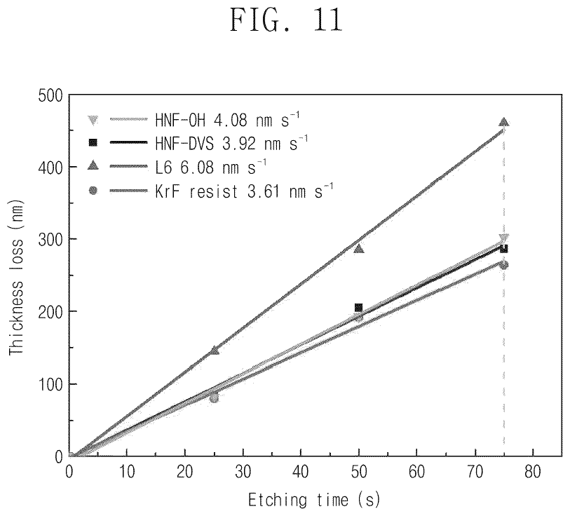

FIG. 11 is a graph comparing the etching rates of photoresist thin layers formed according to Experimental Example 6. Referring to FIG. 11, as a result of measuring the etching rates of the first to fourth photoresist thin layers according to Experimental Example 6, the etching rates of the third photoresist thin layer of Comparative Example 1 (L6) and the fourth photoresist thin layer of Comparative Example 2 (KrF resist) were about 6.08 nm/sec and about 3.61 nm/sec, respectively, and the etching rates of the first photoresist thin layer formed by using the resist compound (HNF-OH) and the second photoresist thin layer formed by using the resist compound (HNF-DVS) were about 4.08 nm/sec and about 3.92 nm/sec, respectively. It could be confirmed that the resist compounds (HNF-OH and HNF-DVS) according to the Examples of the inventive concept had better etching resistance than Comparative Example 1 (L6) and had the similar level of the etching resistance as Comparative Example 2 (KrF resist).

The first and second photoresist thin layers formed in Experimental Example 6 were thin layers not undergone an exposing process. If an exposing process is performed on the first and second photoresist thin layers, the first and second photoresist thin layers may include a combined structure of single molecules represented by Formula 1. In this case, the resist compounds (HNF-OH and HNF-DVS) according to the Examples of the inventive concept are expected to achieved better etching resistance than Comparative Example 2 (KrF resist).

In Experimental Example 6, the resist (L6) disclosed in Korean Registration Patent No. 10-2215511 of Comparative Example 1 includes a material represented by Formula 7.

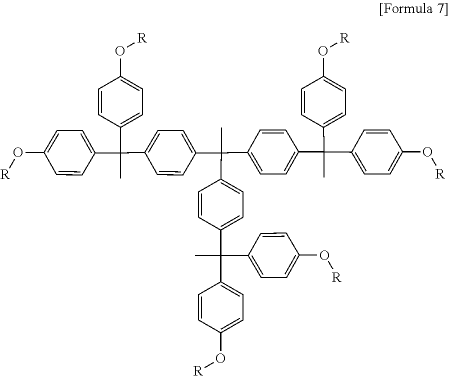

In Formula 7, R is a functional group represented by Formula 8.

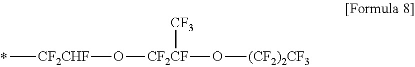

In Formula 8, * is a part bonded to the oxygen of Formula 7.

According to the inventive concept, the resist compound may have an organic single molecular structure represented by Formula 1, and a photoresist layer may include the resist compound. By performing an exposing process and a developing process on the photoresist layer, a photoresist pattern may be formed. Since the resist compound includes the organic single molecular structure represented by Formula 1, the sensitivity and resolution of the photoresist pattern may be improved, and the etching resistance of the photoresist pattern may be increased. Further, the developing process may be performed using a fluorine-based solvent as a developing solution, and accordingly, the pattern collapse of the photoresist pattern may be minimized.

Accordingly, a resist compound capable of improving the resolution and sensitivity of a photoresist pattern, increasing the etching resistance of a photoresist pattern, and restraining the collapse of a photoresist pattern, a method for forming the same, and a method for manufacturing a semiconductor device using the same, may be provided.

Although the embodiments of the present invention have been described, it is understood that the present invention should not be limited to the embodiments, but various changes and modifications can be made by one ordinary skilled in the art within the spirit and scope of the present invention as hereinafter claimed.

What is claimed is:

1. A resist compound for photolithography, represented by the following Formula 1: 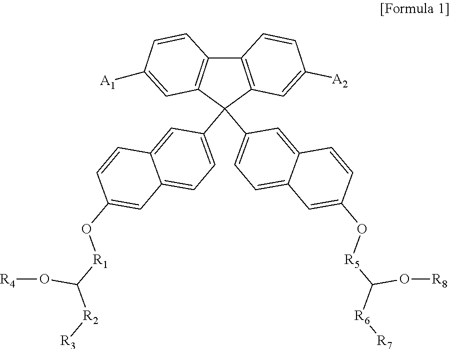
in Formula 1, R₁ and R₅ are each independently an alkylene group of 1 to 4 carbon atoms, R₂ and R₆ are each independently a single bond or an alkylene group of 1 to 4 carbon atoms, R₃ and R₇ are each independently a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms or a fluoroaryl group of 6 to 20 carbon atoms, R₄ and R₈ are each independently hydrogen, deuterium, or a vinyl silyl group, and A₁ and A₂ are hydrogen or an aryl group of 6 to 20 carbon atoms.

2. The resist compound for photolithography of claim 1, wherein, in Formula 1, R₃ and R₇ are each independently a functional group represented by the following Formula 2-1, Formula 2-2, Formula 2-3, or Formula 2-4: 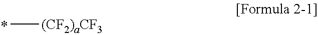
in Formula 2-1, “a” is an integer of 1 to 19: 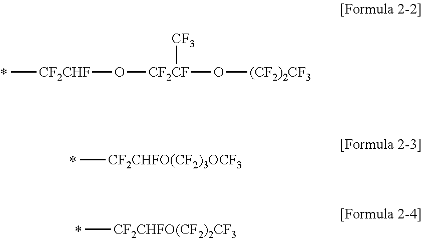
in Formula 2-1 to Formula 2-4, * is a part bonded to the carbon of Formula 1.

3. The resist compound for photolithography of claim 1, wherein, in Formula 1, R₄ and R₈ are each independently a functional group represented by the following Formula 3: 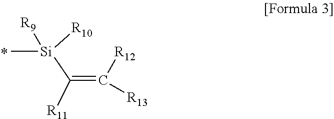
in Formula 3, R₉, R₁₀, R₁₁, R₁₂ and R₁₃ are each independently hydrogen, deuterium, or an alkyl group of 1 to 3 carbon atoms, and * is a part bonded to the oxygen of Formula 1.

4. The resist compound for photolithography of claim 1, wherein the material represented by Formula 1 comprises a material represented by the following Formula 4: 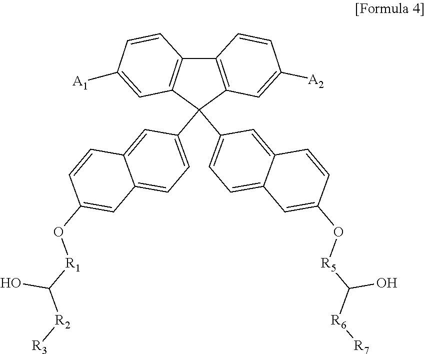
in Formula 4, R₁, R₂, R₃, R₅, R₆, R₇, A₁ and A₂ are the same as defined in Formula 1.

5. The resist compound for photolithography of claim 1, wherein the material represented by Formula 1 comprises a material represented by the following Formula 5: 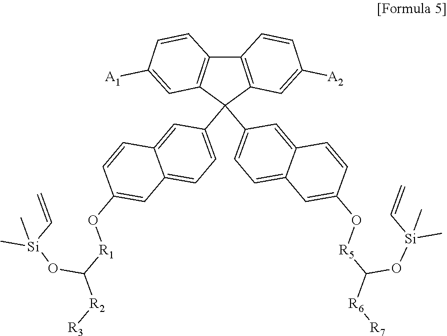
in Formula 5, R₁, R₂, R₃, R₅, R₆, R₇, A₁ and A₂ are the same as defined in Formula 1.

6. A method for forming a resist compound for photolithography, the method comprising:
providing a single molecule having a core structure including at least six benzene rings; and
combining a functional group with the core structure, wherein
the combining of the functional group with the core structure comprises combining a preliminary functional group including a fluorine-containing group and a hydroxyl group (—OH) with the core structure,
the combining of the preliminary functional group with the core structure comprises performing S_{N}2 type ring-opening reaction using the single molecule having the core structure, and an oxygen-containing heterocyclic compound of 2 to 5 carbon atoms, having the fluorine-containing functional group, and
the preliminary functional group is combined with any one among the benzene rings of the core structure.

7. The method for forming the resist compound for photolithography of claim 6, wherein the oxygen-containing heterocycle compound is an epoxide.

8. The method for forming the resist compound for photolithography of claim 6, wherein the combining of the functional group with the core structure, further comprises substituting the hydroxyl group (—OH) of the preliminary functional group with a vinyl silyl group.

9. The method for forming the resist compound for photolithography of claim 6, wherein
the oxygen-containing heterocyclic compound is an epoxide, and
the S_{N}2 type ring-opening reaction using the single molecule having the core structure and the epoxide having the fluorine-containing functional group is performed according to the following Reaction 2-1 or Reaction 2-2: 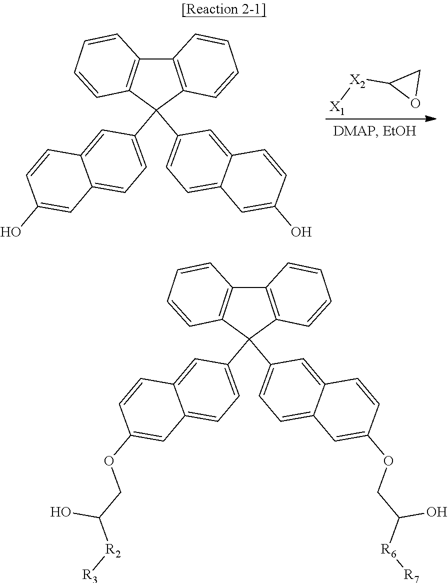 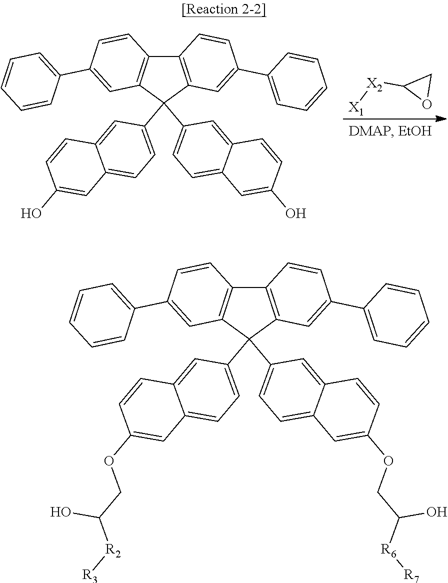
in Reaction 2-1 and Reaction 2-2, X₁, R₃ and R₇ are the fluorine-containing functional groups, X₁, R₃ and R₇ are each independently a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, or a fluoroaryl group of 6 to 20 carbon atoms, X₂, R₂ and R₆ are each independently a single bond or an alkylene group of 1 to 4 carbon atoms, DMAP is 4-dimethylaminopyridine, and EtOH is ethanol.

10. The method for forming the resist compound for photolithography of claim 9, wherein the combining of the functional group with the core structure, further comprises substituting the hydroxyl group (—OH) of the preliminary functional group with a vinyl silyl group, and
the substitution of the hydroxyl group (—OH) of the preliminary functional group with the vinyl silyl group is performed according to the following Reaction 3-1 or Reaction 3-2: 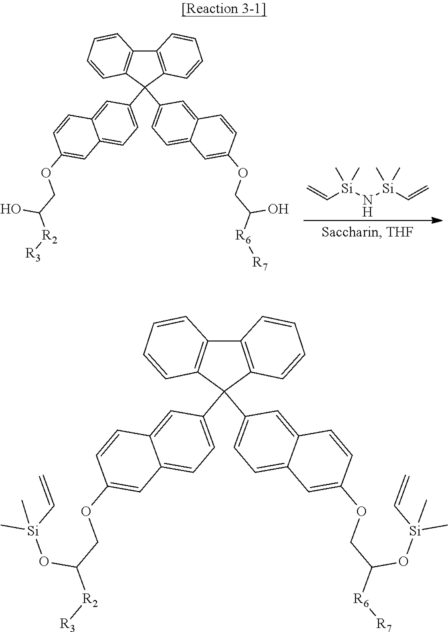 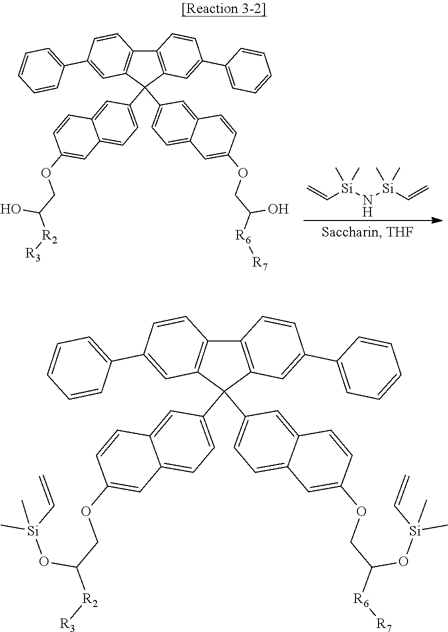
in Reaction 3-1 and Reaction 3-2, R₂, R₃, R₆ and R₇ are the same as defined in Reaction 2-1 and Reaction 2-2, and THF is tetrahydrofuran.

11. A method for manufacturing a semiconductor device, the method comprising:
forming a lower layer on a substrate; and
forming a photoresist layer on the lower layer r, wherein
the photoresist layer comprises a resist compound having an organic single molecular structure,
the resist compound comprises a core structure including at least six benzene rings, and a functional group combined with at least one of the benzene rings of the core structure, and
the functional group comprises at least one among a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms, a fluoroaryl group of 6 to 20 carbon atoms, and a vinyl silyl group.

12. The method for manufacturing the semiconductor device of claim 11, wherein the resist compound is represented by the following Formula 1: 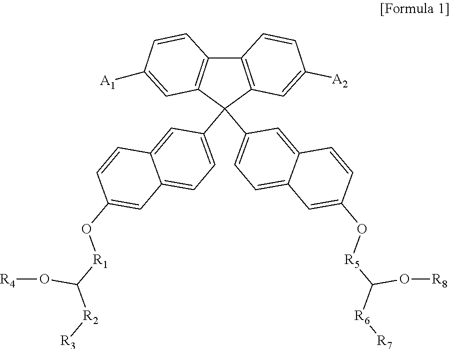
in Formula 1, R₁ and R₅ are each independently an alkylene group of 1 to 4 carbon atoms, R₂ and R₆ are each independently a single bond or an alkylene group of 1 to 4 carbon atoms, R₃ and R₇ are each independently a fluoroalkyl group of 1 to 20 carbon atoms, a fluoroalkyl ether fluoroalkyl group of 2 to 20 carbon atoms, a fluoroalkyl ether fluoroalkylene ether fluoroalkyl group of 3 to 20 carbon atoms or a fluoroaryl group of 6 to 20 carbon atoms, R₄ and R₈ are each independently hydrogen, deuterium, or a vinyl silyl group, and A₁ and A₂ are hydrogen or an aryl group of 6 to 20 carbon atoms.

13. The method for manufacturing the semiconductor device of claim 12, further comprising performing an exposing process on the photoresist layer, wherein
the photoresist layer comprises a first part exposed by the exposing process and a second part unexposed by the exposing process, and
the exposing process is performed using e-beam or extreme ultraviolet.

14. The method for manufacturing the semiconductor device of claim 13, wherein, in the first part of the photoresist layer, the resist compound comprises carbon radicals produced by the irradiation of the e-beam or extreme ultraviolet, and
in the first part of the photoresist layer, the resist compound comprises a combined structure of the molecules represented by Formula 1 by the combination reaction of the carbon radicals and the vinyl silyl group.

15. The method for manufacturing the semiconductor device of claim 14, wherein in the second part of the photoresist layer, the resist compound comprises a single molecular structure represented by Formula 1.

16. The method for manufacturing the semiconductor device of claim 13, further comprising performing a developing process to selectively remove the second part of the photoresist layer, wherein
the developing process is performed using a developing solution including a fluorine-based solvent.

17. The method for manufacturing the semiconductor device of claim 12, wherein the material represented by Formula 1 comprises a material represented by the following Formula 4: 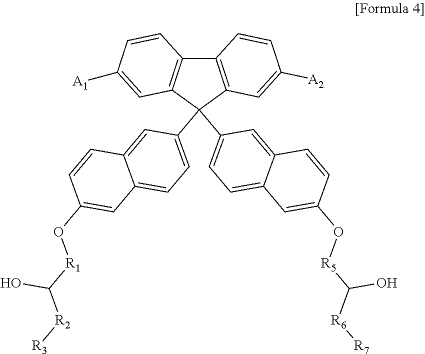
in Formula 4, R₁, R₂, R₃, R₅, R₆, R₇, A₁ and A₂ are the same as defined in Formula 1.

18. The method for manufacturing the semiconductor device of claim 12, wherein the material represented by Formula 1 comprises a material represented by the following Formula 5: 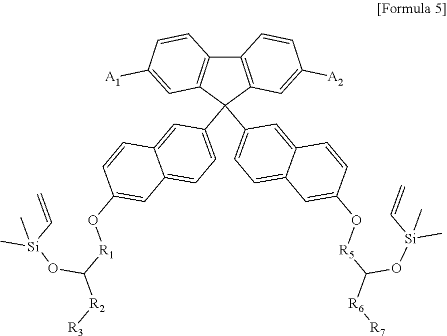
in Formula 5, R₁, R₂, R₃, R₅, R₆, R₇, A₁ and A₂ are the same as defined in Formula 1.

19. The method for manufacturing the semiconductor device of claim 12, wherein, in Formula 1, R₃ and R₇ are each independently a functional group represented by the following Formula 2-1, Formula 2-2, Formula 2-3, or Formula 2-4: 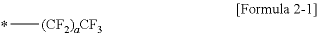
in Formula 2-1, “a” is an integer of 1 to 19: 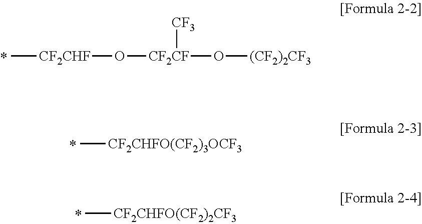
in Formula 2-1 to Formula 2-4, * is a part bonded to the carbon of Formula 1.

20. The method for manufacturing the semiconductor device of claim 12, wherein, in Formula 1, R₄ and R₈ are each independently a functional group represented by the following Formula 3: 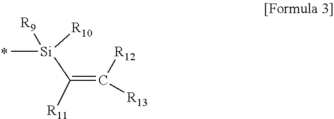
in Formula 3, R₉, R₁₀, R₁₁, R₁₂ and R₁₃ are each independently hydrogen, deuterium, or an alkyl group of 1 to 3 carbon atoms, and * is a part bonded to the oxygen of Formula 1.
